(19) United States
(12) Patent Application Publication
(10) Pub. No.: US 2021/0331274 A1
(43) Pub. Date: Oct. 28, 2021

(54) METHODS FOR LASER COATING OF SILICON NITRIDE ON A METAL SUBSTRATE

(71) Applicant: SINTX TECHNOLOGIES, INC., Salt Lake City, UT (US)

(72) Inventors: Bryan J. McEntire, Salt Lake City, UT (US); Bhajanjit Singh Bal, Salt Lake City, UT (US); Ryan M. Bock, Salt Lake City, UT (US)

(21) Appl. No.: 17/239,412

(22) Filed: Apr. 23, 2021

Related U.S. Application Data
(60) Provisional application No. 63/014,235, filed on Apr. 23, 2020.
(60) Provisional application No. 63/040,802, filed on Jun. 18, 2020.

(51) Int. Cl. B23K 26/342; B23K 26/12; C23C 24/10

(52) U.S. Cl. CPC B23K 26/342; A61L 27/06; C23C 24/10; B23K 26/123

(57) ABSTRACT

Various embodiment related to methods for coating a metal substrate with a silicon nitride ceramic coating are disclosed herein. The metal substrate may be a biomedical implant with a laser-cladded silicon nitride coating for promoting osteogenesis.

### CROSS-REFERENCE TO RELATED APPLICATIONS

This application claims priority to U.S. Provisional Application No. 63/014,235; filed Apr. 23, 2020, and U.S. Provisional Application No. 63/040,802, filed Jun. 18, 2020, the contents of which are entirely incorporated by reference herein.

### FIELD

The present disclosure relates to systems and methods for silicon nitride laser cladding, and in particular to improving the biological response of metal substrates using silicon nitride laser cladding.

### BACKGROUND

Zirconium oxide or “zirconia” (ZrO₂) is the strongest among the ceramic biomaterials on the market for the fabrication of crowns and fixed partial dentures. Zirconia's natural white color and high mechanical properties make it the ideal candidate material for the production of resistant, esthetically attractive implants.

In the recent past, many concerns have been raised about the suitability of zirconia for biomedical applications, in particular after the catastrophic failure of a series of zirconia femoral heads manufactured in 2001; about 400 femoral heads failed over a short period due to an unexpected, accelerated ageing process caused by a small variation of the process parameters applied during sintering. The unexpected and unprecedented accident raised concerns about other ZrO₂ components; the ISO 13356 (1997) guidelines did not consider material aging at the time.

At room temperature, zirconia is only stable in its monoclinic form, which has relatively low mechanical properties. When heated above 1170° C., monoclinic zirconia transforms into the more compact tetragonal phase, which then inevitably disintegrates by cracking upon cooling. To maintain the integrity of sintered zirconia components, one can either sinter at low temperature, to obtain a fully monoclinic body, or stabilize the tetragonal phase by alloying, thereby avoiding the t-m transformation during cooling. The high fracture toughness exhibited by tetragonal zirconia at room temperature is associated with a stress-induced t→m transformation which inhibits crack propagation. However, zirconia's fracture toughness is still compromised by prolonged exposure to humid environments, a process referred to as low-temperature degradation (LTD).

Other than esthetics and mechanics, the success of dental implants is also determined by biocompatibility. Zirconia is considered an inert biomaterial, meaning that it has limited interactions with biological environments. In vitro and in vivo testing on zirconia showed no evidence for mutagenic or carcinogenic effects and a low affinity to bacterial plaque, but also limited adhesion to biological tissues, in particular bone. Many different treatments have been proposed in order to improve the biological activity of zirconia and promote its integration in existing biological tissues. These include alloying with active phases such as hydroxyapatite, coatings, surface laser modifications, and texturing.

Titanium is another common biomaterial. It is useful for various biomedical purposes such as total joint arthroplasty, traumatic and compound bone fractures, craniomaxillofacial, and dental implants. However, its ability to osseointegrate with human bone is also limited without first functionalizing its surface. Various functionalization methods have been developed. The most common is to first roughen the surface of the titanium implant using sandblasting and then subject it to acid etching. This process creates minute cavities in the surface of the metal that allow osteoblasts to initiate mineralization of the metal surface. Another method of functionalizing titanium is to flame-spray a coating of calcium phosphate or hydroxyapatite onto its surface. This is done for orthopaedic hip stems and acetabular cups in total joint arthroplasty. An additional functionalization method is to apply a coating of calcium phosphate or hydroxyapatite using physical or chemical vapor deposition. However, without appropriate functionalization, titanium does not effectively osseointegrate with native bone tissue. Titanium, like most all metals, can be allergenic or toxic to patients, and post-operative metallosis and pseudotumors are commonly reported in medical journals. Other implanted metals such as cobalt-chromium and stainless steel alloys have similar deficiencies.

From a chemical standpoint, an nm-sized passivation layer (mainly titanium oxide) at the surface of Ti-alloy facilitates the adhesion of osteoblasts, thereby promoting implant fixation to living bone. However, the process is not always successful, if instead of bony fixation, a fibrosis develops between the bone and implant surface. The resulting micromotion can lead to pain and the release of toxic metallic ions at the bone/implant interface.

In addition to the failure of bone ingrowth into porous Ti-alloy, periprosthetic infection can also lead to revision, i.e., repeat surgery in joint replacements. The combined incidence from these mechanisms was between 7%-16%, according to Finnish and Danish registry data up to the 2001. Higher fractions (19%-40%) have been reported for patients with degenerative arthritis (depending on age and sex), according to 2013-2017 data from the Canadian registry. Cementless fixation may itself be protective against infection risk. The incidence of periprosthetic infections with cementless Ti-alloy implants after short-term implantation was about half that for other types of implants, according to the Norwegian Arthroplastic Registry for the period 1987-2003. The lack of cement in the total hip joint construct may contribute to a decreased risk of infection. One mechanism for this observation may be the passivation layer on Ti-alloy that partly counteracts bacterial colonization. Electron-hole pairs trapped at the surface layer of oxidized TiO₂ have strong reducing and oxidizing properties. Their reaction with H₂O or hydroxide ions generates hydroxyl radicals (OH) and superoxide ions (O₂) that attack polyunsaturated phospholipids in the bacterial membrane and catalyze site-specific damages in their DNA. Even with this inherent antibacterial mechanism on Ti surfaces, periprosthetic infection remains a concern, and is a leading cause of repeat surgery in joint replacement.

Biopolymers are additional implant materials that have poor osseous integration characteristics. Polyethylene (PE), polyurethane (PU), polymethylmethacrylate (PMMA), polyetheretherketone (PEEK), and polyetherketoneketone (PEKK) are several polymers that require surface functionalization to integrate with native bone. Similar functionalization methods are used for these materials including surface roughening, acid etching, or coating them with titanium, calcium phosphate or hydroxyapatite. While surface roughening and acid etching are helpful in promoting osseous integration, their ability to do so is generally poorer than biometals; and due to the dissimilarity of the materials, coatings on polymers often fail via delamination at the interface between the coating and the polymer.

Therefore, there is a need for alternative surface functionalization of ceramics—including zirconia and zirconia toughened alumina, biometals—including titanium and titanium alloys, stainless steels, and cobalt-chromium alloys, and biopolymers—including polyethylene, polyurethane, polyetheretherketone, and polyetherketoneketone—to promote osteointegration.

### SUMMARY

In an aspect, the present disclosure encompasses a method of A method for coating a metal substrate with a silicon nitride ceramic coating, the method comprising: providing silicon nitride powder within a laser cladding system having a build plat-form and a laser beam source; providing a metal substrate on the build platform, wherein the metal substrate; providing a constant flow of a gas within the laser cladding system; spreading a layer of a Si₃N₄ powder over the metal substrate; pulsing a laser beam from the laser source over the metal substrate and the layer of Si₃N₄ powder to form a silicon nitride coating on the metal substrate; and repeating the previous steps until the silicon nitride coating has a continuous thickness of 15±5 μm.

In various aspects, the Si₃N₄ powder comprises a mixture of: 90 wt. % silicon nitride (β-Si₃N₄) which has been previously reacted with 6 wt. % yttrium oxide (Y₂O₃) and 4 wt. % aluminum oxide (Al₂O₃) to form a β-SiYAlON composite structure. In an aspect, the metal comprises titanium. In some aspects, the metal comprises biomedical grade commercially pure titanium. In some aspects, the gas comprises nitrogen gas. The nitrogen gas may limit silicon nitride oxidation and decomposition.

In some aspects, the laser beam has a wavelength of 1064 nm, is pulsed with a spot size of 2 mm, is driven by an applied voltage of 400 V, and/or is pulsed at a pulse time of 4 ms.

In an aspect, the silicon nitride coating has greater osteogenic activity than the metal substrate without the silicon nitride coating. In additional aspects, the silicon nitride coating has greater antibacterial properties than the metal substrate without the silicon nitride coating.

Other aspects and iterations of the invention are described more thoroughly below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of this specification, illustrate several embodiments of the invention and together with the description serve to explain the principles of the invention.

FIG. 1 is a schematic representation of the deposition process.

FIG. 2A shows the surface morphology of polished zirconia before laser cladding, obtained by laser microscopy.

FIG. 2B shows the surface morphology of polished zirconia after laser cladding, obtained by laser microscopy.

FIG. 2C shows the surface morphology of roughened zirconia before laser cladding, obtained by laser microscopy.

FIG. 2D shows the surface morphology of roughened zirconia after laser cladding, obtained by laser microscopy.

FIG. 3 shows surface morphology of a polished and laser-cladded zirconia sample with a net of buckling cracks clearly visible.

FIG. 4 shows surface roughness (Ra) of uncoated and coated zirconia, measured by laser microscopy.

FIG. 5A shows Si₃N₄ powders observed by SEM at low magnification.

FIG. 5B shows Si₃N₄ powders observed by SEM at high magnification.

FIG. 5C is a cross sectional analysis shown in an isometric view.

FIG. 5D is a distribution of the powder equivalent diameter measured by SEM.

FIG. 6A is a scanning electron image of a roughened zirconia sample before laser cladding at low magnification.

FIG. 6B is a scanning electron image of a roughened zirconia sample before laser cladding at high magnification.

FIG. 6C is a scanning electron image of a roughened zirconia sample after laser cladding at low magnification.

FIG. 6D is a scanning electron image of a roughened zirconia sample after laser cladding at high magnification.

FIG. 7 shows Raman spectra of both zirconia substrates, the laser-cladded layer and stoichiometric Si₃N₄ as a reference.

FIG. 8A shows a composition of the laser-cladded surface obtained in the region of Si2p.

FIG. 8B shows a composition of the laser-cladded surface obtained in the region of O1s.

FIG. 8C shows a composition of the laser-cladded surface obtained in the region of N1s.

FIG. 8D shows a composition of the laser-cladded surface obtained in the region of Zr3d.

FIG. 8E is a quantification of FIGS. 8A-8D.

FIG. 9 shows XRD crystallographic patterns obtained on polished zirconia and on the laser-cladded coating. The inset shows an EBSD image of the cross-section of the coating with Si₃N₄ (yellow) and crystalline Si—Si domains (red) clearly visible.

FIG. 10 shows WST optical density results measured after 10 days on the different samples and Si₃N₄ as a reference.

FIG. 11 shows fluorescence microscopy results obtained on the four different samples, showing the presence and distribution of cell nuclei (blue), osteocalcin (green) and osteopontin (red) on the surfaces.

FIG. 12 shows results of SaOS-2 cell proliferation and bone formation test based on direct cell nuclei (blue), osteopontin (red) and osteocalcin (green) counting of fluorescence-stained microscopic images.

FIG. 13 shows reference Raman spectra of a laser-cladded surface after SAOS-2 testing for 10 days.

FIG. 14 shows collagen maturity ratio for the various samples obtained from the intensity of the Raman bands at 1658 and 1691 cm⁻¹.

FIG. 15 shows carbonate-to-phosphate ratio for the various samples obtained by the intensity of the Raman bands at 1078 and 961 cm⁻¹.

FIG. 16 shows mineral-to-matrix ratio for the various samples obtained by the intensity of the Raman bands at 961 and 1658 cm⁻¹ and at 1078 and 1658 cm⁻¹.

FIG. 17A shows a laser microscope substrate surface image of untreated LDPE.

FIG. 17B shows a laser microscope substrate surface image of coated LDPE.

FIG. 17C shows a laser microscope substrate surface image of untreated Ti6Al4V.

FIG. 17D shows a laser microscope substrate surface image of coated Ti6Al4V.

FIG. 17E shows a laser microscope substrate surface image of untreated ZTA.

FIG. 17F shows a laser microscope substrate surface image of coated ZTA.

FIG. 17G shows a laser microscope substrate surface image of untreated Y-TZP.

FIG. 17H shows a laser microscope substrate surface image of coated Y-TZP.

FIG. 18A shows a SEM secondary electron image overlapped with an EDS compositional maps for an LDPE surface analysis.

FIG. 18B shows a SEM secondary electron image overlapped with an EDS compositional maps for an LDPE cross-section analysis.

FIG. 18C shows a SEM secondary electron image overlapped with an EDS compositional maps for a Ti6Al4V surface analysis.

FIG. 18D shows a SEM secondary electron image overlapped with an EDS compositional maps for a Ti6Al4V cross-section analysis.

FIG. 18E shows a SEM secondary electron image overlapped with an EDS compositional maps for a ZTA surface analysis.

FIG. 18F shows a SEM secondary electron image overlapped with an EDS compositional maps for Y-TZP surface analysis.

FIG. 18G shows a SEM secondary electron image overlapped with an EDS compositional maps for a Y-TZP cross-section analysis.

FIG. 19A shows an optical morphology of the surface of an LDPE substrate with silicon nitride particles.

FIG. 19B shows a Raman imaging map of the surface of an LDPE substrate with silicon nitride particles.

FIG. 19C shows a Raman imaging map of the surface of an LDPE substrate with silicon nitride particles.

FIG. 19D shows a Raman spectra of a silicon nitride coated LDPE substrate.

FIG. 20A shows an optical morphology of the surface of a Ti6Al4V substrate with silicon nitride particles.

FIG. 20B shows a Raman imaging map of the surface of a Ti6Al4V substrate with silicon nitride particles.

FIG. 20C shows a Raman imaging map of the surface of a Ti6Al4V substrate with silicon nitride particles.

FIG. 20D shows a Raman spectra of a silicon nitride coated Ti6Al4V substrate.

FIG. 21A shows a Raman imaging map of the surface of a ZTA substrate with silicon nitride particles.

FIG. 21B shows a Raman imaging map of the surface of a ZTA substrate with silicon nitride particles.

FIG. 21C shows a Raman imaging map of the surface of a ZTA substrate with silicon nitride particles.

FIG. 21D shows a Raman imaging map of the surface of a Y-TZP substrate with silicon nitride particles.

FIG. 21E shows a Raman imaging map of the surface of a Y-TZP substrate with silicon nitride particles.

FIG. 21F shows a Raman imaging map of the surface of a Y-TZP substrate with silicon nitride particles.

FIG. 22 is a qualitative representation of the applied voltage/Si—Si bond relationship measured by X-ray Photoelectron Spectroscopy, as an indicator of substrate chemical composition.

FIG. 23A shows microbial viability assays (WST) of Staphylococcus epidermidis as an indicator of bacterial exposure time for silicon nitride coated and uncoated LDPE substrates.

FIG. 23B shows microbial viability assays (WST) of Staphylococcus epidermidis as an indicator of bacterial exposure time for silicon nitride coated and uncoated Ti6Al4V substrates.

FIG. 23C shows microbial viability assays (WST) of Staphylococcus epidermidis as an indicator of bacterial exposure time for silicon nitride coated and uncoated Y-TZP substrates.

FIG. 24A is a fluorescence micrograph of a LDPE sample showing osteocalcin after exposure to SaOS-2 cells.

FIG. 24B is a fluorescence micrograph of a LDPE sample showing osteopontin after exposure to SaOS-2 cells.

FIG. 24C is a fluorescence micrograph of a LDPE sample showing nuclei after exposure to SaOS-2 cells.

FIG. 24D is a fluorescence micrograph of a silicon nitride coated LDPE sample showing osteocalcin after exposure to SaOS-2 cells.

FIG. 24E is a fluorescence micrograph of a silicon nitride coated LDPE sample showing osteopontin after exposure to SaOS-2 cells.

FIG. 24F is a fluorescence micrograph of a silicon nitride coated LDPE sample showing nuclei after exposure to SaOS-2 cells.

FIG. 25A is a fluorescence micrograph of a Ti6Al4V sample showing osteocalcin after exposure to SaOS-2 cells.

FIG. 25B is a fluorescence micrograph of a Ti6Al4V sample showing osteopontin after exposure to SaOS-2 cells.

FIG. 25C is a fluorescence micrograph of a Ti6Al4V sample showing nuclei after exposure to SaOS-2 cells.

FIG. 25D is a fluorescence micrograph of a silicon nitride coated Ti6Al4V sample showing osteocalcin after exposure to SaOS-2 cells.

FIG. 25E is a fluorescence micrograph of a silicon nitride coated Ti6Al4V sample showing osteopontin after exposure to SaOS-2 cells.

FIG. 25F is a fluorescence micrograph of a silicon nitride coated Ti6Al4V sample showing nuclei after exposure to SaOS-2 cells.

FIG. 26A is a fluorescence micrograph of a Y-TZP sample showing osteocalcin after exposure to SaOS-2 cells.

FIG. 26B is a fluorescence micrograph of a Y-TZP sample showing osteopontin after exposure to SaOS-2 cells.

FIG. 26C is a fluorescence micrograph of a Y-TZP sample showing nuclei after exposure to SaOS-2 cells.

FIG. 26D is a fluorescence micrograph of a silicon nitride coated Y-TZP sample showing osteocalcin after exposure to SaOS-2 cells.

FIG. 26E is a fluorescence micrograph of a silicon nitride coated Y-TZP sample showing osteopontin after exposure to SaOS-2 cells.

FIG. 26F is a fluorescence micrograph of a silicon nitride coated Y-TZP sample showing nuclei after exposure to SaOS-2 cells.

FIG. 27A shows relative surface substrate amounts assigned to the fluorescent probes used for osteocalcin, osteopontin, and cell nuclei based on fluorescence micrograph images of LDPE substrates before and after treatment.

FIG. 27B shows relative surface substrate amounts assigned to the fluorescent probes used for osteocalcin, osteopontin, and cell nuclei based on fluorescence micrograph images of Ti6Al4V substrates before and after treatment.

FIG. 27C shows relative surface substrate amounts assigned to the fluorescent probes used for osteocalcin, osteopontin, and cell nuclei based on fluorescence micrograph images of Y-TZP substrates before and after treatment.

FIG. 28 is a schematic of a laser-cladding fabrication procedure for producing a dense Si₃N₄ ceramic coating on a Titanium substrate.

FIG. 29A is an SEM observation showing the interface between the Si₃N₄ coating and the Titanium substrate, while FIG. 29B is an EDS analysis of Nitrogen, FIG. 29C is an EDS analysis of Silicon and FIG. 29D is an EDS analysis of Titanium that show these elements on a cross-section of a Si₃N₄-coated Titanium substrate.

FIGS. 30A and 30B are low magnification EBSD images taken in the vicinity of the interface between coating and Titanium substrate to image size and orientation of the metal microstructure after laser treatment, while FIGS. 30C and 30D are high magnification EBSD images taken in the vicinity of the interface between coating and Titanium substrate to image size and orientation of the metal microstructure after laser treatment.

FIG. 31A is an image showing a micro hardness indentation made on the cross section of the coating sample and FIG. 31B is a graphical representation showing the average hardness values as a function of distance from the interface between the coating and the substrate.

FIG. 32A is a low magnification of an SEM micrograph taken at the site of a scratch test applied to 25 N on the Si₃N₄-coated surface of a Titanium substrate; and FIG. 32B is a high magnification of an SEM micrograph taken at the site of a scratch test applied to 25 N on the Si₃N₄-coated surface of a Titanium substrate. FIGS. 32C, 32D, and 32E show elemental EDS analyses in the chemical distributions at the scratch site for N, Si, and Ti elements, respectively.

FIG. 33A is a graphical representation of volumes of hydroxyapatite deposited by SaOS-2 cells per unite of substrate; FIG. 33B is a graphical representation of amounts of Gla-osteocalcin and osteopontin on Si₃N₄-coated and uncoated Titanium substrates; FIGS. 33C and 33D show typical laser microscopy images in planar and three-dimensional views, respectively, that were employed in the volumetric laser-microscopy assessments for uncoated Titanium-alloy; FIGS. 33E and 33F show typical laser microscopy images of planar and three-dimensional view, respectively, for Si₃N₄-coated Titanium substrates; and FIGS. 33G and 33H show merged fluorescence images of Gla-osteocalcin (green) and osteopontin (red) in bone tissue grown on uncoated Titanium and Si₃N₄-coated Titanium substrates, respectively.

FIG. 34A is a graphical representation of cell viability of dead/live Gram-positive S. epidermidis at 24 and 48 hours for uncoated and Si₃N₄-coated Titanium substrates; FIG. 34B is a graphical representation of counts of dead/live Gram-positive S. epidermidis at 24 and 48 hours for uncoated and Si₃N₄-coated Titanium substrates; FIGS. 34C, 34D, 34E, and 34F are fluorescence micrographs taken at 48 hours; and FIGS. 34G, 34H, 34I, and 34J are fluorescence micrographs of Si₃N₄-coated substrates.

FIG. 35A is an image of an uncoated Ti6Al4V acetabular shells specifically designed with a porous surface for cementless hip arthroplasty; FIG. 35B is an image of a Si₃N₄-coated Ti6Al4V acetabular shells specifically designed with a porous surface for cementless hip arthroplasty; FIGS. 35C and 35D are images of the uncoated and Si₃N₄-coated acetabular shells, respectively, shown at higher magnification; and FIGS. 35E and 35F are graphical representations showing the quantitative laser microscopy assessments on the structure of a porous structure of the shell before and after being coated with Si₃N₄, respectively.

FIG. 36A is an image showing the results of luciferin “glowing” experiments at 48 hours conducted on Gram-negative E. coli bacteria-contaminated with an uncoated acetabular shell; FIG. 36B is an image showing the results of luciferin “glowing” experiments at 48 hours conducted on Gram-negative E. coli bacteria-contaminated with a Si₃N₄-coated Ti6Al4V acetabular shell; and FIGS. 36C and 36D are images showing an observation of microscopic areas of incomplete coating.

FIG. 37A is a schematic of a osteoblast interactions at the biomolecular interface with Si₃N₄ eukaryotic cells use NH₄⁺ molecules as a nutrient while being able to dissociate NH₃ to ultimately produce NO and use it as a signal for osteoblastogenesis; and FIG. 37B is a schematic of bacteria interactions at the biomolecular interface with Si₃N₄ eukaryotic cells use NH₄⁺ molecules as a nutrient while also being able to dissociate NH₃ to ultimately produce NO and use it as a signal for osteoblastogenesis.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others.

As used herein, the terms “comprising,” “having,” and “including” are used in their open, non-limiting sense. The terms “a,” “an,” and “the” are understood to encompass the plural as well as the singular. Thus, the term “a mixture thereof” also relates to “mixtures thereof.”

As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

As used herein, the term “silicon nitride” includes Si₃N₄, α-Si₃N₄ or β-Si₃N₄, β-SiYAlON, SiYON, SiAlON, or combinations of these phases or materials.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

Provided herein are methods of laser cladding treatment with Si₃N₄ powders that are applied on the surface of a substrate in order to stimulate osteointegration. Laser cladding uses a high-density laser source which melts a feedstock material, usually in the form of wire or powder. The melted material may then be used to produce coatings. Industrial laser sources may reach high power density, so that the feedstock material may be melted in milliseconds without com-promising the properties of the substrate. Alternatively, the laser power may locally melt the surface of the substrate thereby allowing ceramic particles to be embedded into the substrate by the fluence of the laser. This allows for the use of laser cladding on “soft” substrates, such as polymers. For example, laser cladding may be used to deposit bioactive materials, such as silicon nitride, on soft low-melting polymers. Thanks to the high powder density of the laser source, the same technique can also be used to produce bioactive coatings. Cladded coatings can be used to improve osteointegration and prevent infections on substrates.

Further provided herein are various embodiments for a laser-cladding manufacturing method that produces a dense Si₃N₄ ceramic coating on a biomedical grade commercially pure titanium (cp-Ti) substrate by an automatic laser-cladding procedure. In an embodiment, the Si₃N₄-coating may be applied to commercially available Ti-alloy acetabular shells for total hip arthroplasty. In the present disclosure, a novel method of depositing a Si₃N₄ layer on anodized Ti-alloy using an automatic laser-cladding procedure is disclosed.

Silicon nitride has higher mechanical properties (hardness, toughness, resistance to cyclic loading, etc.) than conventional bioceramics and a series of additional bioactive effects which cannot be achieved with oxide-based materials. Silicon nitride may provide resistance to bacterial colonization, combined with the ability to stimulate osteoblast differentiation and production of bone tissue. Without being limited to any one theory, the beneficial effects of silicon nitride may be the result of the formation and release of both nitrogen (NH₃, NH₄⁺) and silicon (Si(OH)₄) species. The nitrogen moieties stimulate cellular proliferation and lyse common bacteria strains; and silicon is converted to silicic acid which actively contributes to the formation of mineralized bone tissue.

Provided herein are biomedical implants or substrates with laser-cladded silicon nitride coatings, methods of coating the surface of a biomedical implant or substrate with silicon nitride using laser cladding, and methods of promoting osteogenesis using the biomedical implant with laser-cladded silicon nitride coating.

The method of coating the surface of a biomedical implant or substrate may include laser cladding a surface of a biomedical implant with silicon nitride powder. In some embodiments, the method may include providing the biomedical implant or substrate, roughening at least one surface of the biomedical implant or substrate, laser cladding a coating of silicon nitride on the roughened surface, and repeating the laser cladding step until the coating has a thickness of at least 10 μm. The laser cladding method may include directing a laser beam to the roughened surface of the biomedical implant or substrate and pre-applying a silicon nitride powder or simultaneously directing a silicon nitride powder to the roughened surface of the biomedical implant or substrate. In one example, laser cladding of silicon nitride on substrates may result in the formation of a composite coating based on silicon nitride particles in a matrix of titanium, and nanocrystalline and amorphous silicon.

Non-limiting examples of materials that may be included in the biomedical implant or substrate include polymers, titanium, titanium alloys, alumina, zirconia, mixtures of alumina and zirconia, stainless steel, and cobalt chromium alloys, polyethylene, polyurethane, polyetheretherketone (PEEK), and/or polyetherketoneketone (PEKK). In various examples, the biomedical implant or substrate may include zirconia-toughened alumina (ZTA), yttria-stabilized zirconia (Y-TZP), titanium (Ti6Al4V), or low- or high-density polyethylene (LDPE, HDPE). In some embodiments, the biomedical implant may be a dental implant, a prosthetic joint, a craniomaxillofacial implant, a bone screw, a bone plate, a bone anchor, an arthrodesis implant such as an intervertebral spinal spacer or a podiatry foot wedge. In at least one example, the biomedical implant may be a dental zirconia substrate. In another example, the biomedical implant may be a titanium prosthetic joint. In another example, the biomedical implant may be a polyetheretherketone spinal spacer. Laser cladding of silicon nitride powders on to zirconia, titanium, or polyetheretherketone substrates may result in the formation of a coarse layer of silicon nitride particles embedded in the ceramic, metal, or polymer substrate.

In an embodiment, the method may include roughening at least one surface of a biomedical implant or substrate. Without being limited to any one theory, roughening the surface may increase the ability of the silicon nitride particles to bond to the surface. The roughening may include abrading the surface of the substrate. In some examples, the substrate may be scratched in a linear pattern, a grid pattern, or at random. The scratches may be formed by diamond abrasive or a glass-cutter diamond blade. In one example, the blade may have a diameter of about 5 μm to about 500 μm. In some embodiments, roughening at least one surface of the biomedical implant may include forming a first set of unidirectional scratches on the at least one surface, rotating the biomedical implant by about 90°, and forming a second set of unidirectional scratches perpendicular to the first set of unidirectional scratches. The scratches may be about 5 μm to about 500 μm wide. In various examples, the scratches may be about 5 μm to about 50 μm, about 5 μm to about 10 μm, about 10 μm to about 20 μm, about 20 μm to about 30 μm, about 30 μm to about 40 μm, about 40 μm to about 50 μm wide, about 50 μm to about 100 μm wide, about 100 μm to about 200 μm wide, about 200 μm to about 300 μm wide, about 300 μm to about 400 μm wide, or about 400 μm to about 500 μm wide. In at least one example, the scratches may be about 25 μm wide. In other embodiments, roughening at least one surface of the biomedical implant may include using a free diamond abrasive or sandblasting by any machine known in the art that may be used to abrade surfaces. The free abrasive machining or sandblasting may be used to form random scratches that may be 5 to 30 μm wide.

In an embodiment, the silicon nitride powder may include α-Si₃N₄, β-SiYAlON, SiAlON, or SiYON. The silicon nitride powder used in the laser cladding process may be formed from a two-phase microstructure including acicular β-Si₃N₄ grains separated by a continuous SiYON grain-boundary phase. The silicon nitride powder may be mechanically ground to an average particle size of about 1 μm to 15 μm.

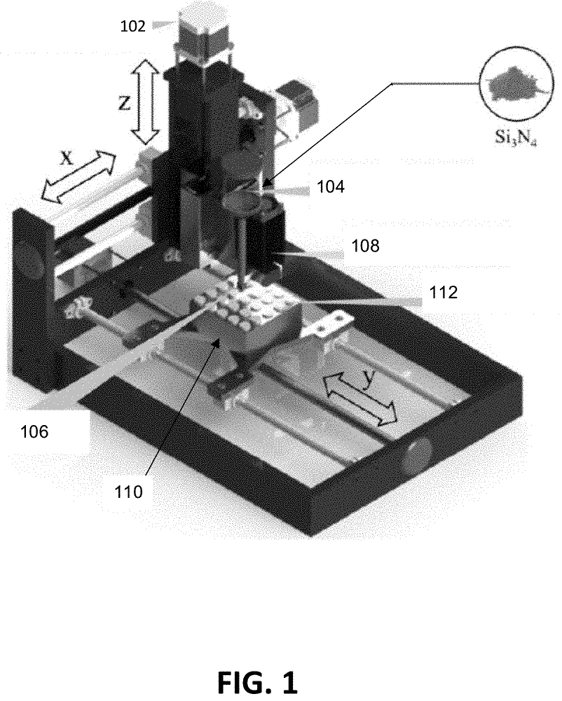

In an embodiment, the powder may then be applied to the roughened substrate surface using laser cladding. FIG. 1 is an exemplary laser cladding system 100 that may be used to apply a silicon nitride coating to a substrate via laser cladding. In some embodiments, the system 100 may include one or more stepper motors 102, a powder tank 104 operable to hold silicon nitride powder, a powder feeder 106, a laser source 108, and a platform 110 operable to hold one or more substrates 112 to be coated with the silicon nitride. The system 100 may be operable to move the platform 110 and/or the powder tank 104 and powder feeder 106 in the x, y, and/or z directions.

Laser cladding may include applying the silicon nitride powder to the surface before or simultaneously with the application of a laser to bond the silicon nitride to the substrate. The laser beam creates a molten pool at the substrate surface, to which the silicon nitride powder is added. The exposure time of the laser on the substrate may be short, such that the cooling is quick. The properties of the laser may be selected such that there is bonding of the silicon nitride powder to the substrate. For example, any combination of laser type, energy and power setting, voltage, pulse and spot size known in the art that achieves the bonding of the silicon nitride powder to the substrate surface may be used. In at least one example, the laser wavelength may be about 1064 nm, have a max pulse energy of about 70 joule, a peak power of about 17 kW, a voltage range of about 160-500 V, a pulse time of about 1-20 ms, and/or a spot size of about 250-2000 μm. In some embodiments, the laser cladding may further include supplying a constant flux of nitrogen gas at the surface of the implant to limit silicon nitride decomposition and oxidation.

The morphology and stoichiometry of the cladded layer may be a function of the applied power, the amount of which may be dependent on the nature and composition of the substrate material. Without being limited to any one theory, the higher the applied power, the higher the amount of silicon there may be in the laser-cladded silicon nitride coating (e.g. silicon-rich, nitrogen-deficient coating). In particular, the nitrogen content may be present in the laser-cladded coating in a range from about 42 at. % to about 70 at. %. In a silicon rich silicon nitride laser-cladded coating, the coating may have between about 42 at. % to about 56 at. % nitrogen. For example, a higher power may lead to a more abundant presence of nanocrystalline silicon in the laser-cladded silicon nitride coating. Silicon plays an important role in the bone formation; in fact silicon ions contribute to the calcification of new bone. For example, the increased silicon ions and the increased surface roughness in claddings on titanium may result in a more homogeneous distribution of cells and bone matrix.

As a non-limiting example, it has been fortuitously found that specific laser power settings and raster speeds lead to appropriate embedding of silicon nitride into titanium. Using a 100 Watt picosecond laser source specifically tuned to emit nanosecond pulses at a raster speed of 5,500 mm/s, it was discovered that one preferred power level and pulse width were 10% to 25% and 20 to 500 μm, respectively. A more preferred power level and pulse width were 15% to 25% and 20 to 500 μm, respectively; and a most preferred power level and pulse width were 25% and 200 to 500 μm, respectively. In addition to the power and pulse width settings, it was also fortuitously discovered that the laser pulse frequency, hatching distance, hatching overlap, and distance of impact all played important roles in obtaining a preferred silicon nitride coating. Hatching distance is the separation of the lines as they are put down (orthogonal to laser beam path) and distance of impact is the center-to-center separation of the individual pulse locations parallel to beam path. A preferred range for pulse frequency is from 110 to 1000 kHz, whereas a most preferred frequency is 1000 kHz. A preferred hatching distance ranges from 0.03 to 0.05 mm, whereas the most preferred hatching distance is 0.03 mm. The most preferred hatching overlap is 60.34%. The preferred distance of laser impact is 0.05 to 0.0055 mm whereas the most preferred distance is 0.0055 mm.

The application of the laser and powder may be repeated at least 1, at least 2, at least 3, at least 4, or up to 5 times in order to obtain a homogenous coating. In some examples, the laser-cladded coating of silicon nitride may have a thickness of at least 5 μm, at least 10 μm, at least 15 μm, at least 20 μm, at least 25 μm, or at least 30 μm. The coating may include about 5 wt. % to about 15 wt. % silicon nitride. In one aspect, the present method allows for Si₃N₄ coatings to be prepared with thicknesses from one to the tens of microns. In one particular embodiment, a coating thickness, t=15±5 μm, may be selected based on projections of homogeneity and scratching resistance.

In some embodiments, the Si₃N₄ coating may meet a 20 N threshold, according to standard scratch testing (ASTM C1624-05). The Si₃N₄ coating may impart both antibacterial and osteogenic properties of bulk Si₃N₄ to the substrate. A fluorescence “glowing” test based on luciferase gene transformation was applied to visualize the colonization of gram-negative Escherichia coli on Si₃N₄-coated and uncoated Ti-alloy acetabular shells. Unexpected results from this test showed that the coating technology conferred resistance to Staphylococcus epidermidis and Escherichia coli adhesion. The scratch resistance of this coating and validation of its osteogenic and antibacterial properties in vitro, with uncoated Ti-alloy and bulk Si₃N₄ serving as negative and positive controls, respectively, were tested. In one aspect, the commercial feasibility of this technology was demonstrated by applying the Si₃N₄ coating to commercially available Ti-alloy acetabular cups, and testing their bacterial resistance in toto.

The cladding coating may be effective for osteointegration. The cladded layers may contribute to bone formation and may provide a variable degree of protection against gram-positive bacteria, in particular for Ti6Al4V substrates. In an embodiment, a method of promoting osteogenesis may include contacting the biomedical implant or substrate with a laser-cladded silicon nitride coating with human tissue. Without being limited to any one theory, the altered composition (sub-stoichiometric nitrogen content) and the crystallographic structure of the cladding may lead to a reduction of the cellular proliferation and surface colonization when compared to monolithic materials. Surprisingly, the silicon nitride cladding induced the formation of a significantly higher amount of bone tissue than the substrate without cladding, even if they had similar values of cell proliferation, and also had a higher surface colonization when observed with fluorescence microscopy.

In some embodiments, bone tissue production increases on the silicon nitride laser-cladded biomedical implant as compared to an implant without the laser-cladded silicon nitride coating. For example, the silicon nitride laser-cladded biomedical implant may have increased osteocalcin and osteopontin distributions, the bone tissue may have a higher degree of cross-linking, and/or may have increased mineralized tissue such as an increase in mineral hydroxyapatite on the surface of the implant. The laser-cladded coating contributes to the stimulation of bone tissue. For example, when compared to uncoated zirconia, silicon nitride cladding may provide improved cellular adhesion and bone tissue formation, with higher degrees of maturity and overall better quality parameters as measured by Raman spectroscopy. The flexibility of the laser cladding technology, based only on a Si₃N₄ powder feeder and a laser beam source, makes this technology suitable also for complex component designs.

### EXAMPLES

### Example 1

### Silicon Nitride Laser Cladding of Yttria-Stabilized Zirconia Samples

### Sample Production

Yttria-stabilized zirconia samples containing 3% of yttria were obtained from a commercial producer. Polished and powdered silicon nitride discs (12 mm diameter, 1 mm thickness) were provided by SINTX Corp. The material consisted of a two-phase microstructure including acicular β-Si₃N₄ grains separated by a continuous and sub-micrometer sized film of Si—Y—O—N grain-boundary phase. To obtain the ceramic powder, coarse powder was mechanically ground to an average particle size of 15 μm.

To produce a “roughening effect” on the otherwise smooth zirconia substrates, the surface was abraded using a glass-cutter diamond blade (tip diameter: 25 μm) under an applied load of 20±5 N. Once the surface was covered by unidirectional scratches, the scratching direction was rotated by about 90° and the operation was repeated.

A schematic of the overall laser-cladding procedure and system is presented in FIG. 1. The conditions applied to achieve a Si₃N₄-cladding were the following: laser wavelength 1064 nm, max pulse energy: 70 joule, peak power 17 kW, voltage range 400 V, pulse time 4 ms, and spot size 2 mm. The apparatus operated under a constant flux of nitrogen gas in order to limit Si₃N₄ decomposition and oxidation. The operation was repeated 3 times in order to obtain a homogenous coating with a thickness of 15±5 μm, over the entire surface of the substrate. A motorized x-y stage with a lateral precision of 10 μm was used to align the sample to the laser source.

### Microscopy and Spectroscopy

Micrographs were taken using a 3D laser-scanning microscope with magnifications ranging from 10× to 150× and a numerical aperture between 0.30 and 0.95. The microscope used an automated x-y stage and an autofocus function for the z range, allowing the acquisition of composite images. The surface roughness values were obtained at 20× magnification, and an average of 10 measurements were performed on areas of 500×500 μm.

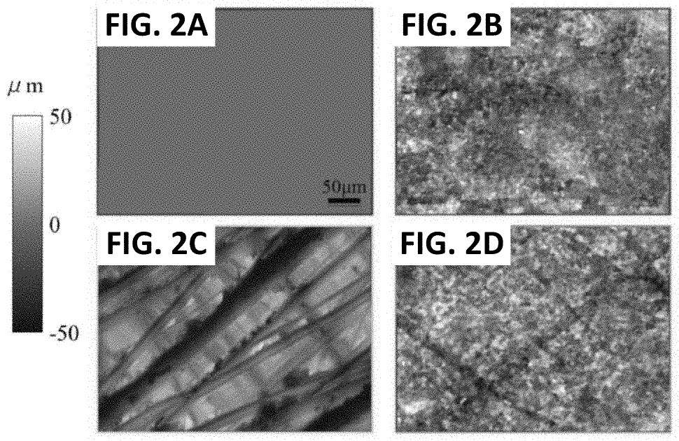

FIGS. 2A-2D shows the morphology of the surface of the pristine polished zirconia surface (FIG. 2A) compared to the roughened substrate (FIG. 2C) and the correspondent surfaces after the application of the Si₃N₄ laser-cladded coating (FIG. 2B and 2D). The laser-cladding process produced a coarse surface where partially embedded Si₃N₄ powder particles are still clearly visible, resulting in the formation of two different orders of roughness: a “macroscopic” local variability of the coating thickness and a microscopic roughening due to the protrusion of powder particles. The roughening process resulted in the formation of oriented scars with a depth of up to about 50 μm that are clearly visible after the application of the coating (FIG. 2D).

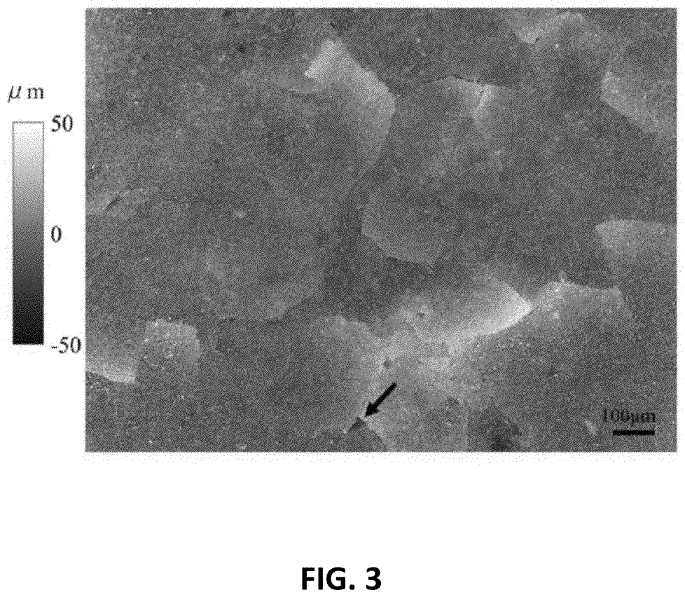

At lower magnifications, a network of cracks was observed on the coated polished zirconia samples (FIG. 3). The shape and topography of the cracks are likely associated with the presence of a buckling phenomenon caused by compressive stresses on the coating. It was observed that the cracks did not result in massive coating delamination, as only small portions of material were missing at the intersections of three or more ridge cracks (black arrow). Compressive residual stresses occurring during solidification are likely due to the difference in coefficient of thermal expansion for the various phases. The application of the roughening process ahead of cladding was helpful to reduce the stress concentration and improve coating adhesion, thereby preventing crack formation.

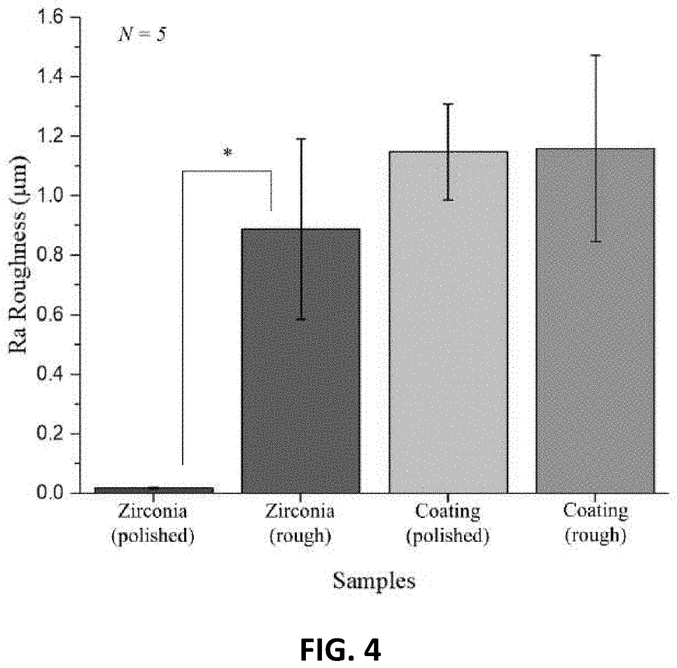

FIG. 4 shows the surface roughness values measured on the different samples using a magnification of 50×. It was observed that the roughening process resulted in values comparable to those achieved after laser-cladding. There was no statistically relevant difference in the surface roughness for the laser-cladded coatings applied to either the polished or surface-roughened zirconia substrates.

A field-emission-gun scanning electron microscope was used to observe and characterize the surface of the samples, before and after cell culture. All images were collected at an acceleration voltage of 10 kV and magnifications ranging between 100× and 50,000×. All samples were sputter-coated with a thin (20 to 30 Å) layer of platinum to improve their electrical conductivity. Crystallographic analyses were performed using a scanning electron microscope (SEM) equipped with an electron-backscattered X-ray diffraction (EBSD) detector.

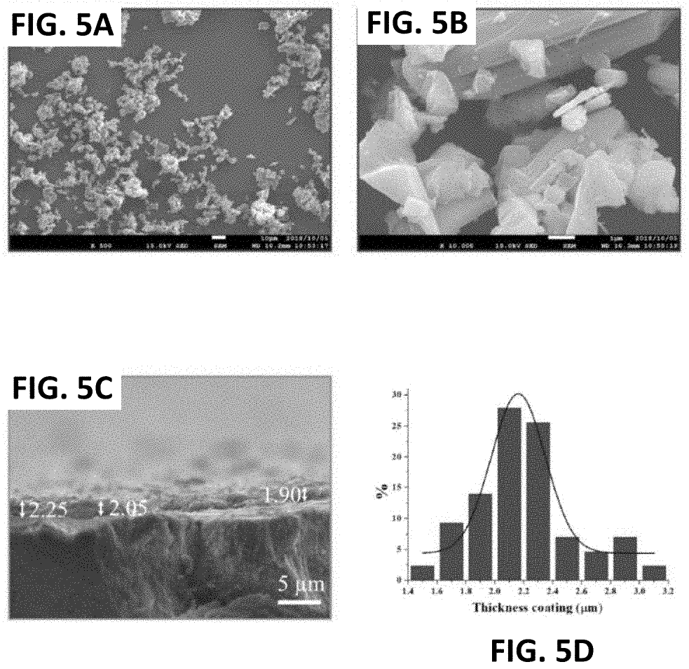

FIGS. 5A and 5B show the SEM images of the Si₃N₄ powders used for the cladding process. It was observed that the grains were sharp and polygonal, from about 300 nm up to a few μm. FIG. 5C shows an isometric view of the coating cross section, with a few, representative thickness values obtained using SEM software. The thickness distribution, from averaging 250 single measurements, is presented in FIG. 5D.

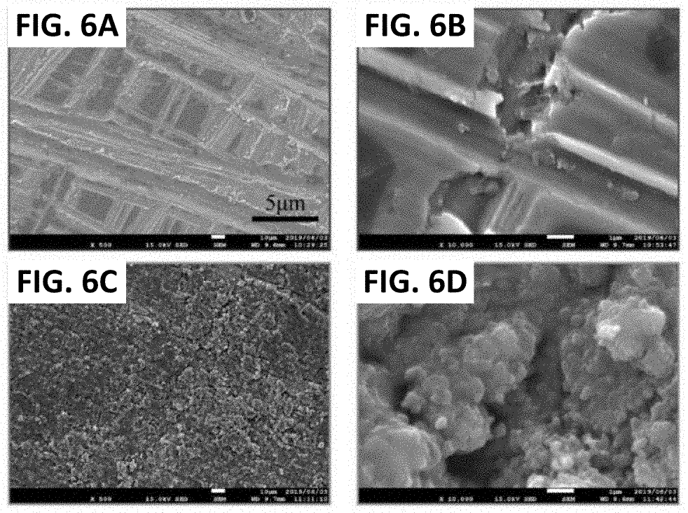

FIGS. 6A-6D show the surface of the roughened zirconia substrates before and after laser-cladding at different magnifications. In FIG. 6A the scratches have two main orientations with a difference of almost 90°. This was selected as the best condition to achieve uniform adhesion of the coating and avoid preferential orientation. At higher magnifications (FIG. 6B), it was observed that the roughening process resulted in the formation of small debris, the amount of which roughly correlated with the depth of the relative abrasion. For the coating, low magnification analysis (FIG. 6C) showed a generally good coverage of the substrate, with only localized defects that look like small cracks or inter-granular voids. At higher magnifications (FIG. 6D), the coating revealed a composite structure with a dispersion of micron and sub-micron globular particles protruding from the surface and forming a classical “cauliflower morphology”.

Raman spectra were collected at room temperature using a triple monochromator equipped with a charge-coupled device (CCD) detector. The spectra were analyzed by commercially available software. The excitation source in the present experiments used a 532 nm Nd:YVO4 diode-pumped solid-state laser operating with a nominal power of 200 mW. A confocal pinhole with an aperture-diameter of 100 μm was placed in the optical circuit to shallow the probe to the order of few μm in depth by excluding photons scattered from out-of-focus regions in the irradiated volume. The lateral resolution of the Raman micro-probe was on the order of 1 μm. An automated, two axes sample stage was employed, making it possible to record spectral maps at given depths by focusing above (or below) the sample surface, and to map spectra with high lateral resolution. For each sample, 25 randomized locations were investigated, and the resulting spectra averaged. All spectra were post-processed removing the baseline and reducing noise with a moving average filter. After fitting to Gaussian curves, the intensity of specific bands (at 1658 and 1691 cm⁻¹ for collagen, 1070 cm⁻¹ for carbonate apatite and 961 cm⁻¹ for phosphate apatite) were used to evaluate the collagen maturity, the phosphate/carbonate ratio and the mineral to matrix ratio.

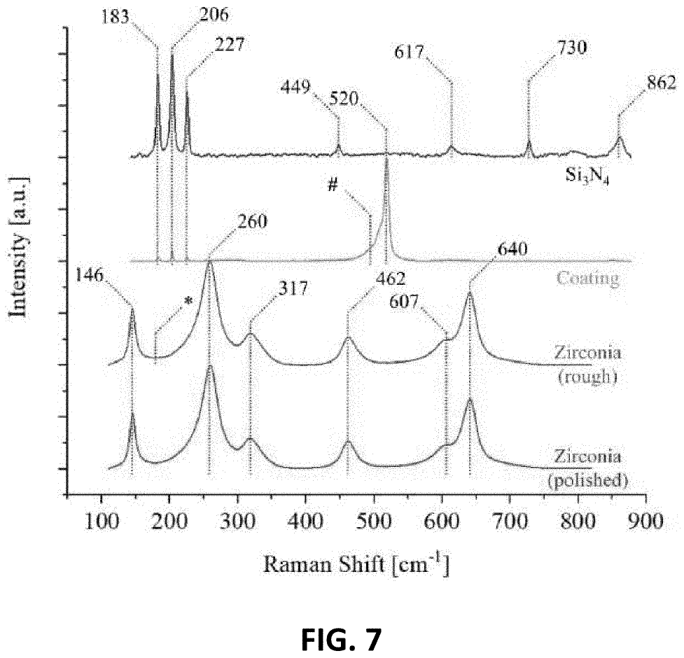

The Raman spectra of the different samples are presented in FIG. 7. The polished substrates were found to be 100% tetragonal zirconia. After roughening, two small peaks related to monoclinic zirconia appeared at about 180 and 192 cm⁻¹ (marked with a “*”). The total amount of monoclinic zirconia was estimated by Raman Spectroscopy using the Katagiri equation. As expected, the value was 0% in the polished areas between scratches, but reached values up to 3% as a result of the diamond blade scratches. The average value on the whole surface was 0.55±0.22%.

The spectra of the coated samples were dominated by a strong band at about 520 cm⁻¹ which is associated to Si—Si vibrations. The asymmetry of the band (to lower Raman shifts) was caused by the presence of a region of sub-bands (marked as “#”), which resulted from nano-crystalline domains of silicon with varying average diameters trapped inside the amorphous silicon matrix. Due to their smaller Raman cross-section, residual Si—N bonds were only observed in the three bands in the region between 150 and 250 cm⁻¹, as confirmed by a comparison with the reference spectrum of stoichiometric β-Si₃N₄.

Analysis of the composition of the laser-cladded silicon nitride layer on the zirconia by Raman spectroscopy showed the presence of a series of sub-bands close to the peak related to Si—Si bonds at 520 cm⁻¹. Even if these sub-bands indicate the presence of sub-micron grains, Raman scattering alone could provide unambiguous information regarding the crystallite size, fraction and distribution. Additional investigations performed using electron backscattered X-ray diffraction were also not able to resolve the crystal structure of the matrix of the laser-cladded layer either, supporting the amorphous/nano-crystalline hypothesis.

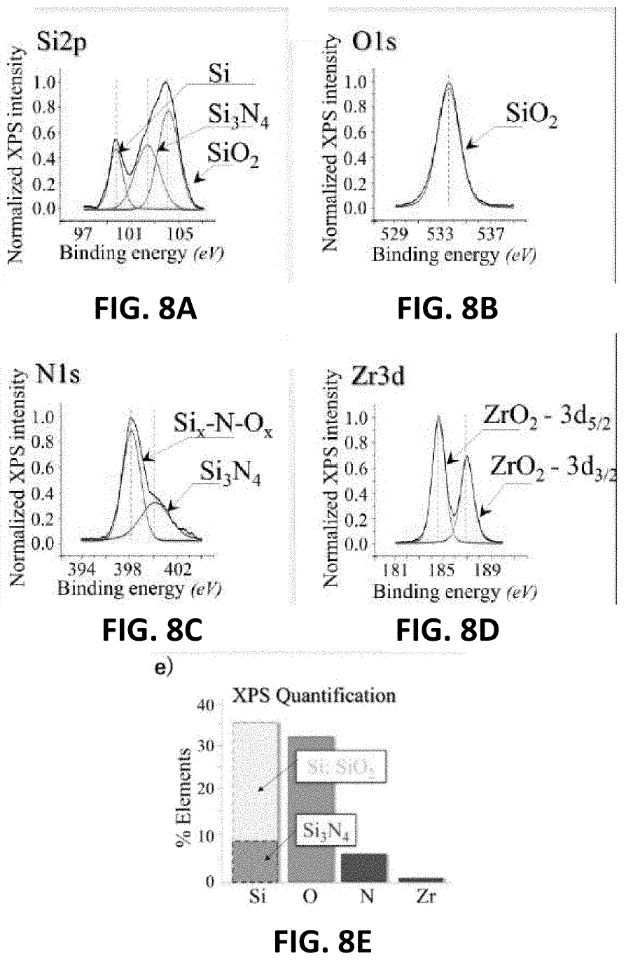

FIGS. 8A-8D shows the XPS spectra of silicon, oxygen, nitrogen and zirconium measured on the coated samples. FIG. 8E is a quantification of FIGS. 8A-8D. The signal of silicon was de-convoluted in three main bands: metallic silicon (100 eV), silicon nitride (102.5 eV) and silicon oxide (104 eV). It was observed that silicon nitride accounts for only about 28% of the silicon content, silicon oxide being the strongest signal, as also confirmed by the spectrum at about 533.5 eV and related to oxygen (1s) bonds. As the deposition was performed under a constant nitrogen flux, the possible initial sources of oxygen were limited to gas trapped in the powders or chemically bound to the silicon nitride in the form of silicon oxide or silicon oxynitride. After deposition, the outermost layer of metallic silicon (Si—Si) spontaneously reacted with the humid atmosphere, resulting in the formation of a thin, uniform layer of silicon oxide. The presence of silicon-nitrogen-oxygen bonds was also confirmed by the spectra of nitrogen (1s) at about 400 eV. Two main peaks were observed: stoichiometric silicon nitride (400 eV) and Six-N—Ox phases, resulting from partial oxidation (398 eV). Finally, all zirconium ions detected by XPS (185-187 eV) appeared to be bonded with oxygen, meaning that the only possible source for such a signal was the residual exposed substrate.

XPS analysis gave further insight to the composition and chemical structure of the layer. As it was observed, only a fraction of the silicon was bonded to nitrogen atoms. Most of it was oxidized, probably due to the exposure to the environment during deposition. An intermediate phase of silicon oxynitride was observed at around 398 eV (N1s). These findings support the bonding structure of silicon oxynitride prepared by oxidation of Si-rich silicon nitride and the spontaneous formation of mixed nitrides/oxides phases when exposed to an oxidizing environment.

X-ray Diffraction (XRD) analysis were performed using a benchtop MiniFlex 300/600 diffractometer equipped with a Cu source, in a 2θ/θ configuration. The 2θ range was comprised between 10 and 90° with a step of 0.01°.

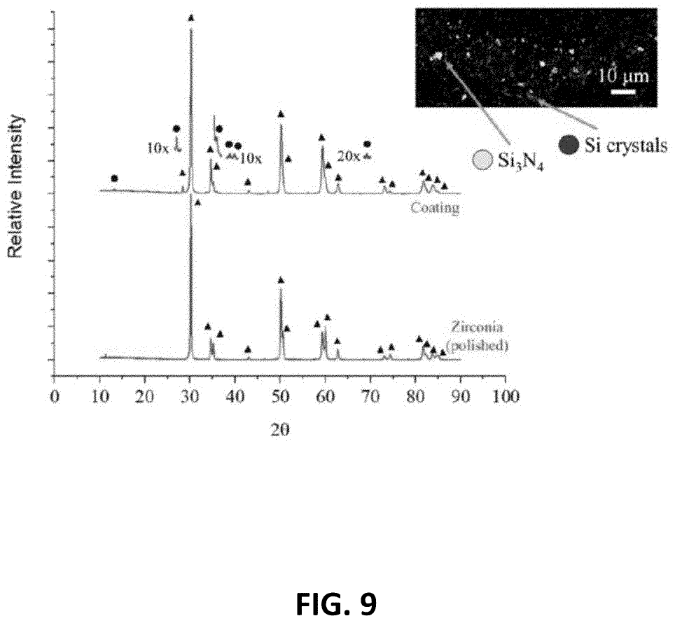

The XRD patterns of the zirconia substrate and coated sample are presented in FIG. 9. The zirconia was observed to be the pure tetragonal phase. When the coating was applied, the overall morphology of the diffraction pattern was essentially unaltered, with a variation in the relative intensity of the two peaks at about 35°, at 60° and again at 84°, which indicated a preferential orientation of the grains. When the diffraction pattern was intensified by a factor 10× or by a factor 20×, secondary bands related to β-Si₃N₄ appeared. No other contribution from the coating was observed in FIG. 9, suggesting that the layer is actually mostly amorphous or nano-crystalline silicon. The results of a cross-sectional analysis performed by EBSD (inset) confirmed the presence of micron and sub-micron crystals of Si₃N₄ (yellow) dispersed in the matrix, further supporting the hypothesis that the coating was mostly formed by an amorphous or nano-crystalline phase.

### Biological Testing and Assays

SaOS-2 human osteosarcoma cells were cultured and incubated in 4.5 g/L glucose DMEM supplemented with 10% fetal bovine serum. The cells were then proliferated in petri dishes for 24 h at 37° C. After adjusting the final cell concentration at 5×10⁵ cell/ml, the cultured cells were deposited on the surface of Si₃N₄-coated and uncoated ZTA substrates (n=3 each) previously sterilized by exposure to UV light. Osteoconductivity tests were conducted by seeding the cells in an osteogenic medium (DMEM supplemented with 50 μg/mL ascorbic acid, 10 mM β-glycerol phosphate, 100 mM hydrocortisone, and ˜10% fetal bovine calf serum), and then incubating the samples for 7 days at 37° C. The medium was changed twice during the week of incubation.

To observe and compare the substrates cytotoxicity, the samples were analyzed using a colorimetric assay based on water-soluble tetrazolium. This technique is based on the employment of a colorimetric indicator (WST-8) which produced a water-soluble formazan dye. The amount of the formazan dye generated was directly proportional to the number of living micro-organisms. Solutions were analyzed using micro-plate readers after collecting OD values for living cells.

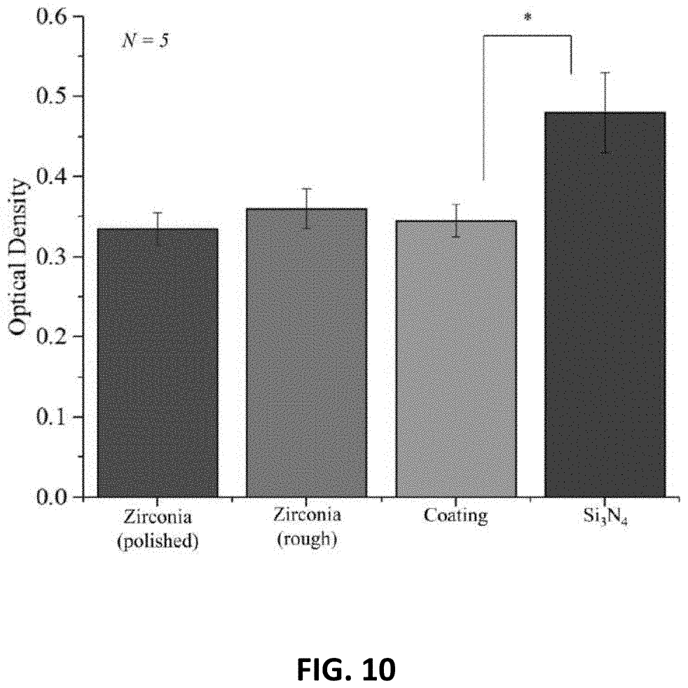

FIG. 10 shows the results of the viability tests performed with the use of a WST-8 assay. It was observed that the amount of cells on the zirconia samples and the laser-cladded surface after 10 days of exposure were similar. However, the optical density measured on the silicon nitride reference sample was significantly higher, meaning that more cells had colonized its surface. The different cellular response to the laser-cladded coating when compared to stoichiometric Si₃N₄ is due to different amounts of available nitrogen on the surface.

Because the structure of the laser-cladded layer is completely different from the base feedstock material, the cellular response of SaOS-2 osteosarcoma deviates from what was previously observed for stoichiometric or nitrogen annealed silicon nitride. Biological assay testing performed using water-soluble tetrazolium showed that while stoichiometric silicon nitride efficiently stimulates cell proliferation, laser cladding is comparable to bioinert materials such as zirconia.

After exposure to osteoblasts, each batch of samples was observed using fluorescence microscopy. Prior to examination, the sample surfaces were treated with different immunostaining reagents, including Hoechst 33342, anti-Human Osteocalcin Clone 2H9F11F8, Isotype IgG, Rabbit polyclonal antibody. Hoechst 33342, a cell nucleus stain, served to visualize cell proliferation, while the other two antibodies were used to stain matrix proteins osteocalcin and osteopontin, respectively, whose concentration quantifies the process of mineralization and bone matrix formation. Subsequently, a secondary antibody, Goat anti-Mouse IgG1 Antibody FITC Conjugated was added to enhance signal detection and visualization. Images were collected using 4× magnification and subsequently analyzed by imaging software in order to count the pixels related to the presence of the different stains.

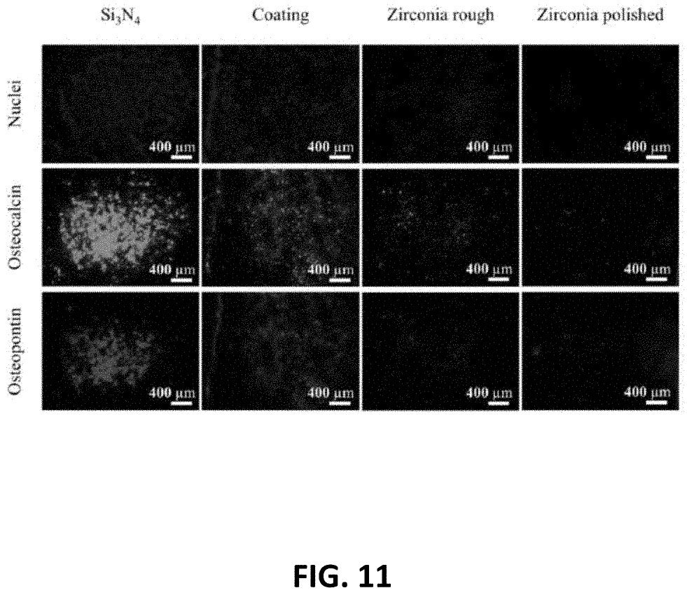

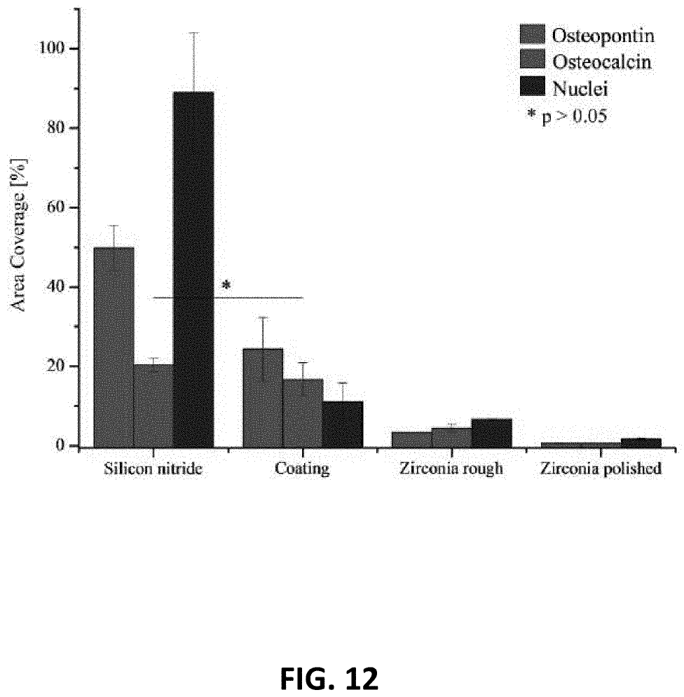

FIG. 11 shows the results of fluorescence microscopy on samples treated for 10 days with SaOS-2 osteosarcoma cells. Stoichiometric silicon nitride was used as a positive control. It was observed that the proliferation of cells on silicon nitride was higher than on zirconia. Roughened samples also generally showed a higher cellular adhesion when compared to smooth surfaces, but the coated zirconia was the only sample able to show a homogeneous distribution of cell nuclei across the entire surface. Moreover, both osteocalcin and osteopontin levels were greater on the coated samples, both smooth and rough zirconia. When compared to stoichiometric silicon nitride, the coated samples showed lower cellular proliferation and, consequentially, lower signals from osteocalcin and osteopontin. The area coverage ratios related to staining were measured and reported in FIG. 12. The results confirmed that the silicon nitride positive control presented the highest amount of cells with the highest synthesis of matrix proteins. The smooth zirconia samples showed the lowest ratios related to mineralized matrix formation.

The fluorescence microscopy images showed increased production of bone tissue for the laser-cladded surface when compared to the pure zirconia samples, as evidenced by both osteocalcin and osteopontin distributions. It must be noted that while both proteins are associated with the presence of bone tissue and their effects is considered synergetic, osteocalcin is mainly observed in mineralized tissue while osteopontin is often associated with bone remodeling. For both, the amount of fluorescence is intermediate between the rough zirconia and the reference silicon nitride sample. These results can be explained by the high bioavailability of silicon from amorphous and nanocrystalline sources when compared to macroscopic crystals.

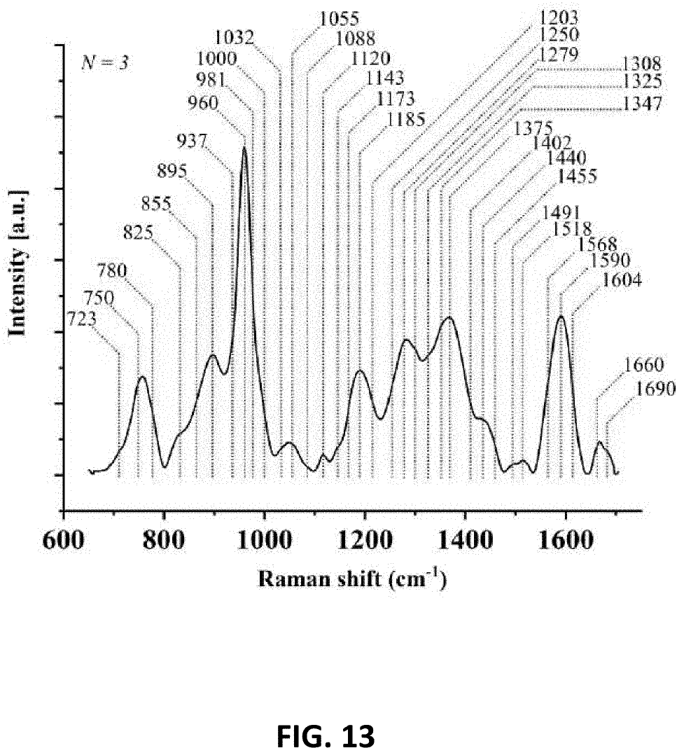

FIG. 13 shows a representative Raman spectrum acquired on the surface of the laser-cladded rough zirconia samples after SaOS-2 osteosarcoma testing in the region between 600 and 1800 cm⁻¹. The region can be roughly divided in four areas. The first region, between 600 and 1000 cm⁻¹, is dominated by a strong peak related to phosphate vibrations, at about 960 cm⁻¹. The second region, between 1200 and 1400 cm⁻¹ is associated with Amide III vibrations. The low-intensity bands in the region between 1400 and 1650 cm⁻¹ are assigned to fatty acids, while the stronger band at 1590 cm⁻¹ is associated with Amide II. Finally, the two relatively weak peaks at 1660 and 1690 cm⁻¹ are the results of Amide I vibrations.

Even if qualitative, the quality parameters obtained by Raman spectroscopy on the bone tissue formed on the laser-cladded layer are comparable to healthy bone and indicate a good degree of maturity, as shown by the values of crosslinking achieved in the bone collagen matrix.

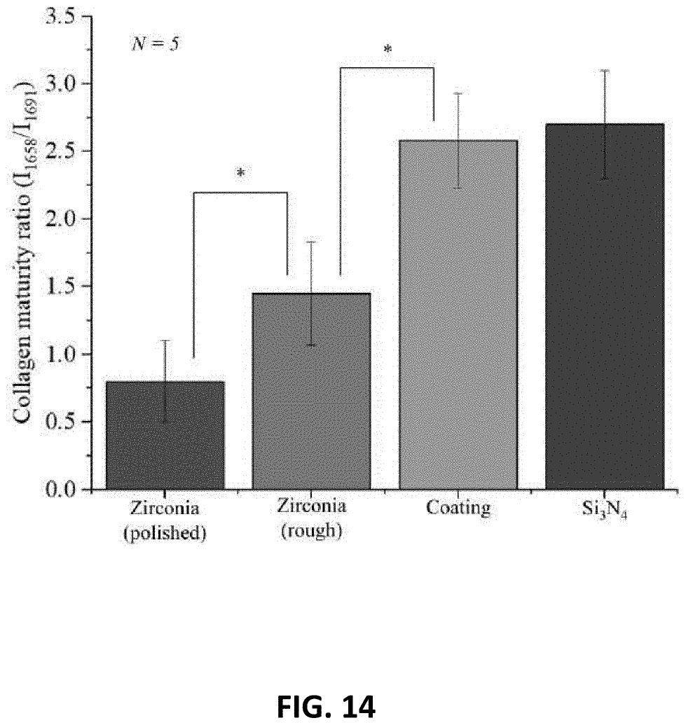

FIG. 14 shows the Collagen Maturity Ratio for the four samples, based on comparing the intensities of the bands at about 1660 and 1690 cm⁻¹. It was observed that the collagen maturity ratio of the bone tissue developed by the osteosarcoma cells on both smooth and rough zirconia was relatively small, representing a low level of cross-linking and a high amount of reducible collagen. Tissue grown on silicon nitride and laser-cladded samples had a higher degree of cross-linking, which is associated with faster osteogenic development.

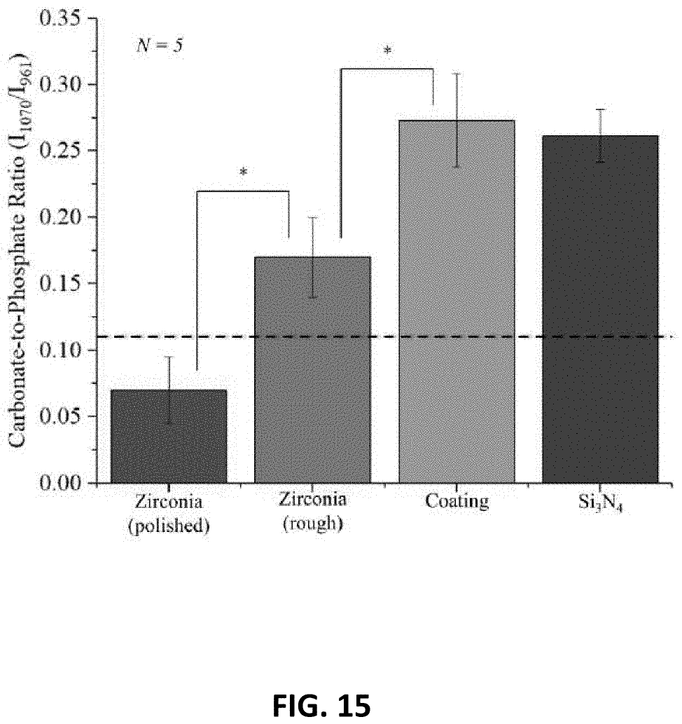

FIG. 15 shows an estimation of the bone mineral phase quality, expressed as a ratio between the main peak related to carbonates and the peak related to phosphate vibrations, respectively at about 1078 and 960 cm⁻¹. It was observed that the amount of carbonate hydroxyapatite formed on the zirconia substrates was lower than on stoichiometric silicon nitride and the laser-cladded coating. Results for the polished zirconia substrate, in particular, were outside the ideal range of carbonate to phosphate percentage values (7-9% wt.) while the roughened zirconia barely reached the minimum value.

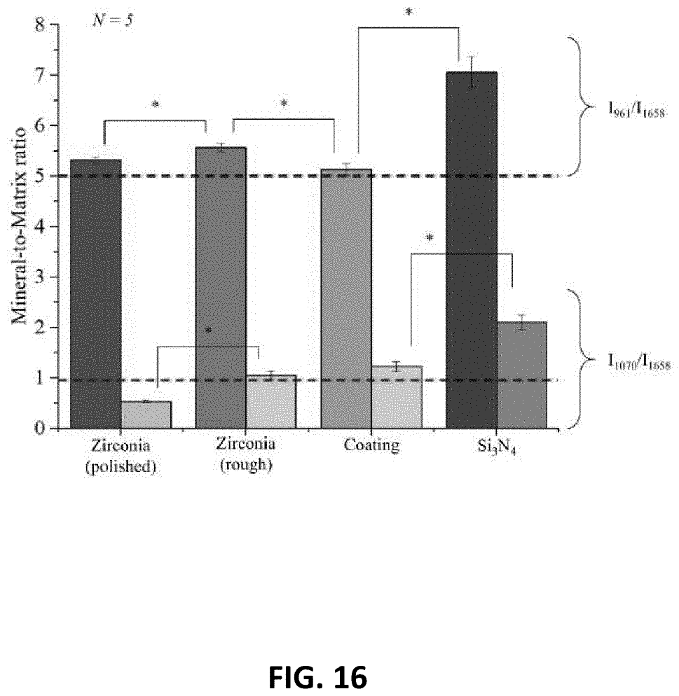

FIG. 16 shows the Mineral to Matrix ratio as estimated by the ratios between phosphate ion vibrations and carbonate ion vibrations with respect to the irreducible collagen at about 1660 cm⁻¹. The minimum value for both ratios is marked with a black dotted line. Three of the four tested specimens meet both requirements, the only exception being the polished zirconia surface, for which the carbonate over irreducible collagen ratio was under the minimum value. The best scores were obtained for the silicon nitride reference, which showed ratios well above the threshold values. Roughened and laser-cladded zirconia showed intermediate values and barely met both requisites, roughened zirconia having more intense phosphate bands and laser-cladded more carbonates.

### Example 2

### Silicon Nitride Laser Cladding of LDPE, Ti6Al4V, ZTA, and Y-TZP

### Sample Production

In this example, Si₃N₄ bulk samples were used as a positive control for biological testing, and were prepared following a procedure previously described. Silicon nitride powder (which had a trimodal distribution with an average size of 0.8±1.0 μm) was obtained from SINTX Technologies Corporation.

Low-density polyethylene (Mw ˜35,000) powder was melted in a vacuum oven at a pressure of 10 Pa and a temperature of 150° C. and molded into 30×50×5 mm plates. The plates were then cut into 10×10×5 mm samples and polished to a roughness of about 500 nm Ra.

Annealed medical grade Ti6Al4V (Al 6%, V 4%, C<0.10%, O<0.20%, N<0.05%, Fe<0.3%) rods with a diameter of 25 mm were cut into 5 mm-thick discs and polished to a roughness of about 500 nm Ra.

Zirconia-toughened alumina (ZTA) samples were obtained by slicing 40 mm diameter CeramTec Biolox® delta femoral heads to obtain 10×10×5 mm block. The cutting was performed with a 2 mm diamond coated blade rotated at 0.5 mm/min to minimize monoclinic zirconia transformation during the procedure. The femoral heads, produced in 2016, had a monoclinic zirconia volume fraction of about 6% before cutting.

Yttria-stabilized zirconia (Y-TZP) samples were obtained from fully densified 3Y-TZP bars (9×4×3 mm) containing 3 mol% of yttria (Y₂O₃) and 0.25 wt. % alumina (Al₂O₃). These samples were fabricated from raw powders using a hot isostatic pressing cycle (for 1 h at 1350° C.) following pressure-less sintering (at 1350° C.), and possessed an average grain size of about 0.2 μm.

A Vision LWI VERGO-Workstation Nd:YAG laser (wavelength of 1064 nm, max pulse energy: 70 joule, peak power 17 kW, voltage range 160-500 V, pulse time 1-20 ms, spot size 250-2000 μm) with an automatic x-y stage (lateral resolution: 10 m) was used to produce a silicon nitride coating. To achieve homogeneous coatings on the various substrate materials, the laser source parameters and the number of layers were adjusted through trial and error before each treatment. The pulse time was optimized to reduce surface overheating (and microstructural changes in titanium) or burning, while the voltage was selected as the lowest value that could grant at least a coverage of about 33% of the surface on the first cladded layer. The optimized parameters are listed in Table 1.

TABLE 1
Deposition parameters for various substrates. LDPE, low-
density polyethylene; Ti6Al4V, titanium; ZTA, zirconia-
toughened alumina; Y-ZTP, yttria-stabilized zirconia.

| | Voltage | Pulse Time | Laser Spot Size | | |
|---|---|---|---|---|---|
| Substrate | (V) | (ms) | (mm) | Layers | Gas |
| LDPE | 225 | 10 | 2 | 1 | N₂ |
| Ti6Al4V | 425 | 10 | 2 | 3 | N₂ |
| ZTA | 325 | 10 | 2 | 3 | N₂ |
| Y-TZP | 250 | 10 | 2 | 3 | N₂ |

A layer of silicon nitride powder of about 50 μm thickness was pre-coated on the surface of the samples and then heated with a 2 mm laser spot size under constant N₂ gas flow (1.5 atm). To cover all the surface substrate, the stage moved on the x-y stage with a step of 1 mm, in order to overlap the single laser spots and create a more homogeneous layer.

For the ZTA, the Y-TZP, and the Ti6Al4V, three cladding layers were required to achieve full coverage. Before depositing the next layer, the substrate was rotated by 90° to form a cross grid. However, only one layer was applied to the LDPE substrate because the laser easily melts and oxidizes polyethylene. Attempts at a second layer resulted in polymer carbonization.

### Sample Characterization

Raman spectra and Raman maps were collected at room temperature using a RAMANtouch instrument with an excitation frequency of 532 nm green line and equipped with a 400 1340-pixel charge coupled device (CCD) camera. All data were analyzed using commercially available software (Raman Viewer, Laser RAMAN Microscope).

Surface morphology was characterized using a confocal scanning laser microscope capable of high-resolution optical imaging with depth selectivity. All images were collected at 50× magnification. The roughness of each sample was measured at 25 random locations.

Scanning electron microscopy (SEM) and energy dispersive X-ray spectroscopy (EDS) were used to acquire high magnification images and sample chemical composition maps.

Surface roughness plays a fundamental role in antibacterial properties and for this reason, the surface morphologies were studied and compared before and after deposition.

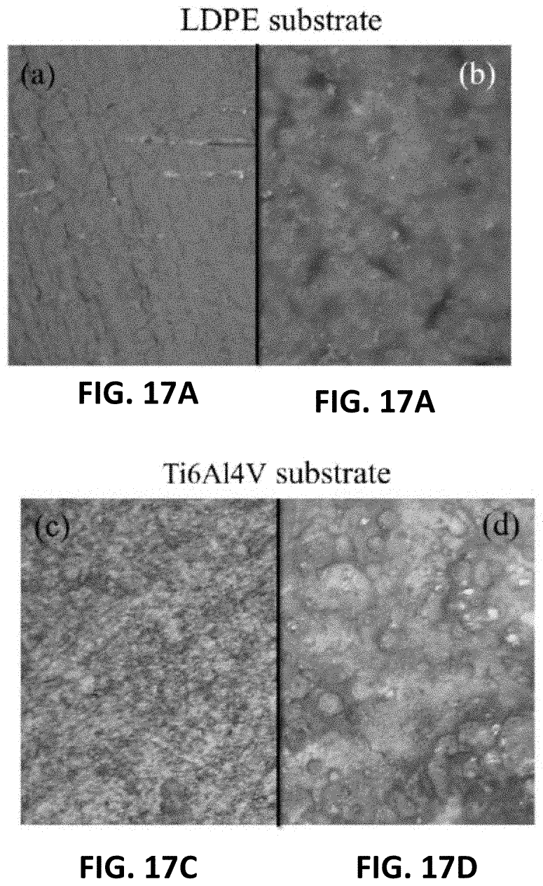

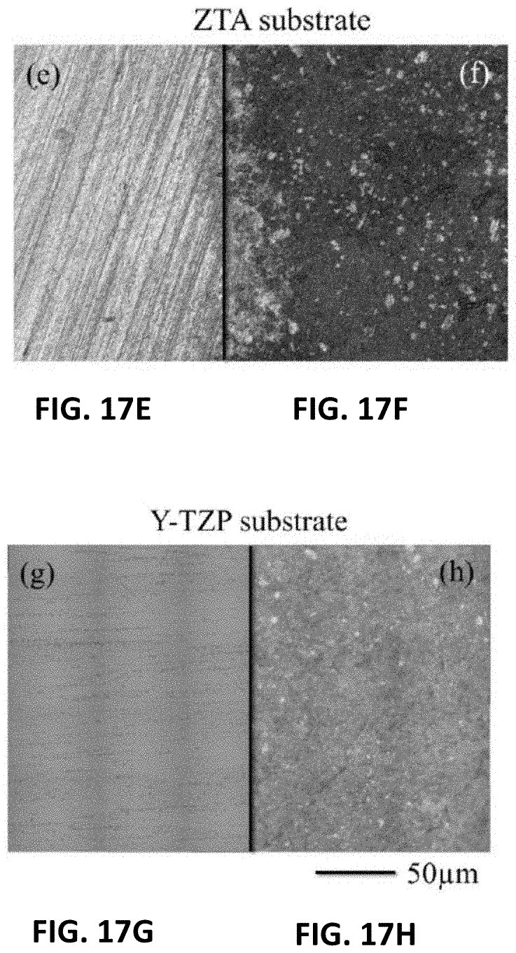

FIGS. 17A-17H show sample surfaces before and after laser cladding, a treatment which increased the surface roughness of all samples. The roughness of the LDPE plate prepared surface finishing was initially in the range of Ra=0.30±0.02 μm (FIG. 17A). It increased to Ra=0.81±0.28 μm after treatment (FIG. 17B).

In the case of Ti6Al4V, the initial surface roughness of Ra=0.63±0.09 μm (FIG. 17C) increased to Ra=11.23±1.11 μm (FIG. 17D), a value much higher than that measured on either ceramic substrate. This effect was caused by localized melting of the Ti6Al4V substrate, which resulted in an alteration of the coating-substrate interface morphology and the formation of an alpha phase.

In the case of ZTA, the roughness changed from Ra=1.02±0.02 μm (FIG. 17E) to Ra=4.59±1.29 μm, due to the presence of partially melted particles. This is clearly visible in FIG. 17F.

Because the Y-TZP test bars were polished prior to laser cladding, their initial surface roughness was lower, Ra=0.02±0.01 μm (FIG. 17G). This increased to Ra=1.27±0.42 μm after laser treatment (FIG. 17H).

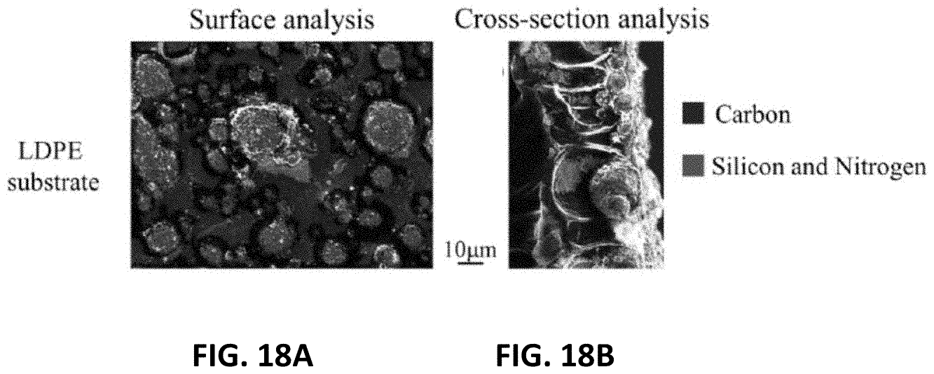

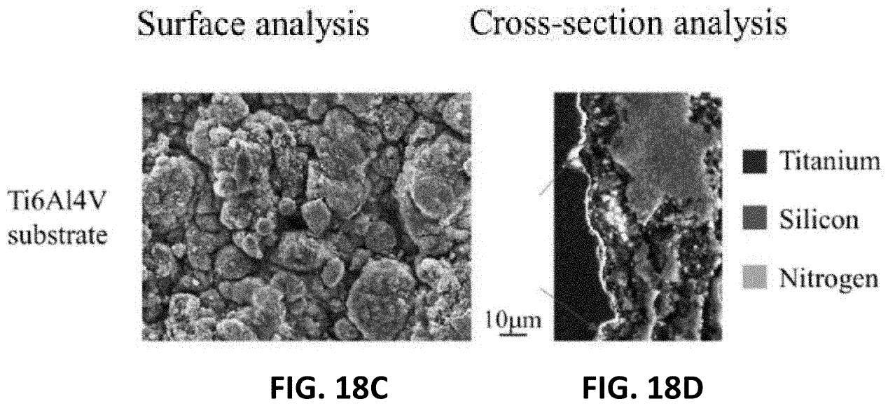

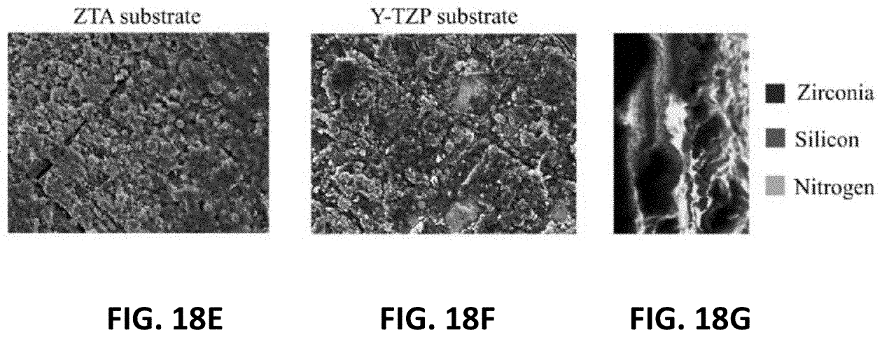

On the LDPE substrate, the LDPE matrix under the Si₃N₄ cladding (FIG. 18A) and on the cross-section images (FIG. 18B), as well as the Si₃N₄ particle penetration inside the LDPE matrix, are visible. Since the silicon nitride particles have a higher absorbance at 1064 nm when compared to LDPE, the ceramic particles heat up and locally melt the polymeric substrate, resulting in the incorporation of silicon nitride into LDPE. As a result, at 225 V the heated particles melt LDPE (at about 120° C.) without causing visible decomposition, which occurs between 350 and 450° C.

On the Ti6Al4V substrate, the silicon nitride coating is more homogeneous, as observed in FIG. 18C. The blue signal (titanium) is still present, but when mixed with the other elements, it changes to a lavender color. On the cross-section of the Ti6Al4V sample (FIG. 18D), it is possible to distinguish between the substrate and the cladded layers. Only occasional formation of Si-Ti intermetallics (see arrow) is evident. Moreover, the coating is composed of two different phases: a silicon-rich (red) layer with a metallic appearance, and a layer with fine dispersion of Si₃N₄ crystals (green) where the nitrogen signal is still intense.

Laser cladding on ceramic substrates resulted in coatings with a similar morphology, composed of partially melted silicon grains (FIGS. 18E and 18F). Due to the high melting point and low surface roughness of the ZTA and Y-TZP substrates, the cladded layer did not strongly bond with the substrate, resulting in partial delamination. Previously, an increase in bonding strength was observed when the substrates were pre-scratched using a diamond tip pen. The cross-section image (FIG. 18G) of a Y-TZP pre-scratched substrate shows a homogeneous layer when compared to both the LDPE and the Ti6Al4V substrates.

Laser treatment converted silicon nitride powder into a silicon phase and also into an amorphous phase. Laser cladding changes the morphology of the substrates to which it is applied, but the final roughness values seem more dependent on the physical properties of the material and applied power than on initial surface roughness. For example, two samples with a similar initial surface roughness, Ti6Al4V and zirconia-toughened alumina (ZTA), showed completely different morphology after treatment. However, initial surface roughness did play a role in cladded layer adhesion, as was observed by comparing the ZTA (rough) and pure zirconia (polished) images. Initially, smooth surfaces resulted in partial delamination of the coating. The applied power may also be correlated with the composition of the cladded layer: higher values resulted in a lower retained nitrogen.

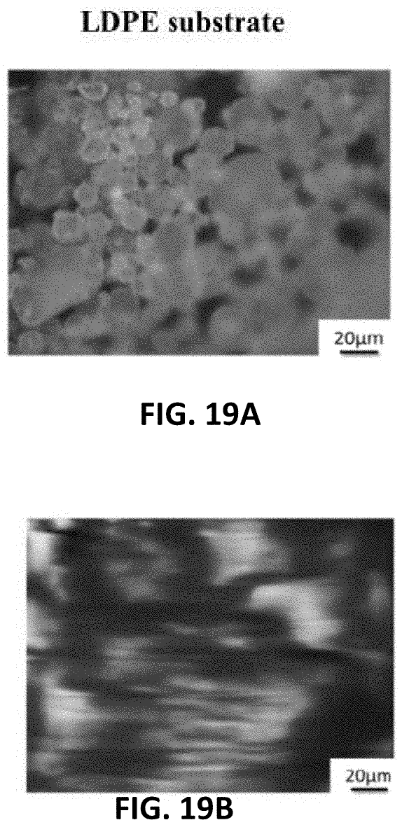

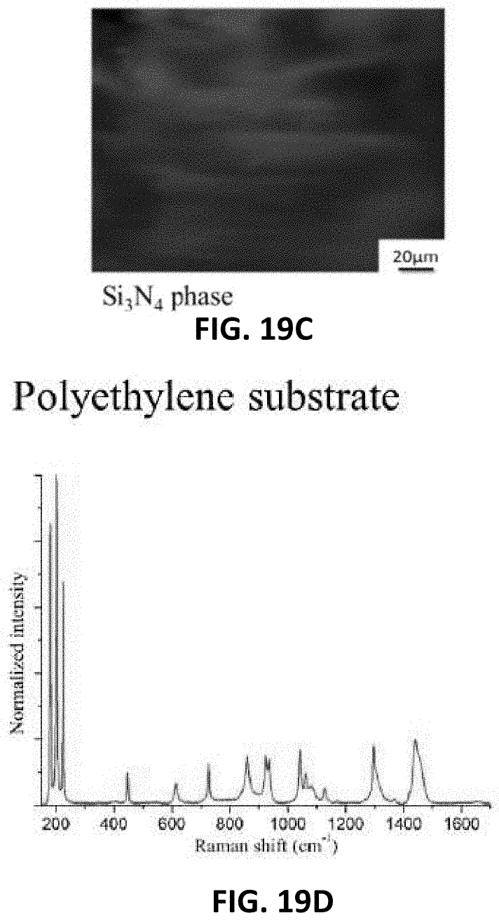

FIGS. 19A-19D show the Raman maps acquired on the LDPE substrate coated with silicon nitride powders. FIG. 19A presents the optical morphology of the surface, with clearly visible silicon nitride particles. The color images were obtained by deconvolution of the Raman signal; FIG. 19B (the yellow map) represents the distribution of the LDPE phase (based on the band at 1300 cm-1) and FIG. 19C (the red map), that of silicon nitride (based on the band at 200 cm-1). The Raman spectra (FIG. 19D) confirmed the presence of silicon nitride particles in the 520 cm⁻¹ region. This was related to the Si—Si bonds. It also confirmed that the composition of the silicon nitride particles was essentially stoichiometric.

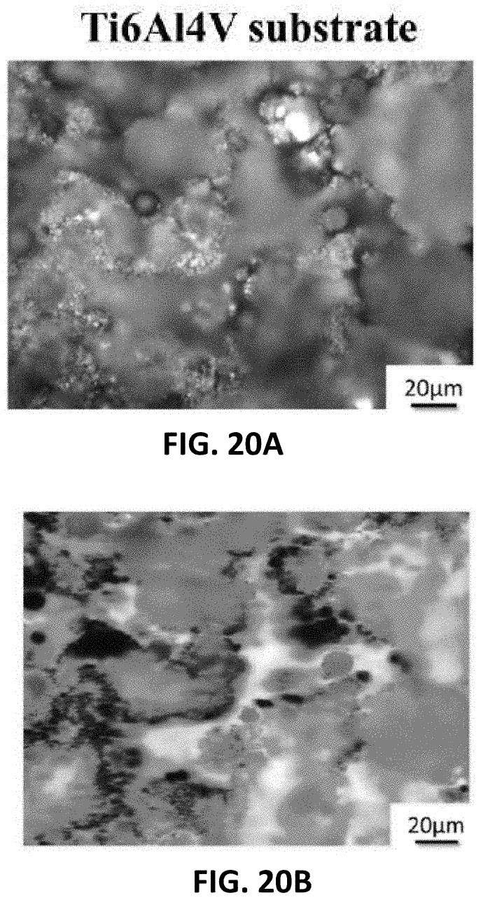

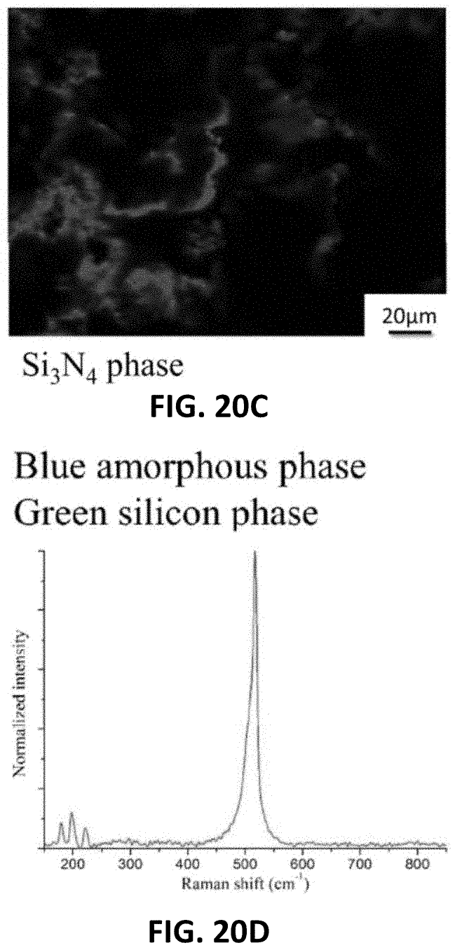

FIGS. 20A-20D show the Raman imaging results acquired on the Ti6Al4V substrate. In this case, the silicon nitride powders underwent degradation during the laser cladding process. FIG. 20A (the blue and green image) shows the distribution of the amorphous silicon phase (shoulder at about 495 cm⁻¹) and the crystalline silicon phase (at about 520 cm⁻¹) (FIG. 20D). FIG. 20C (red image) shows the regions in which the silicon nitride phase is still present, as estimated by the presence of triplet peaks at about 200 cm⁻¹ (FIG. 20D). The red areas correspond to optical image regions in which small silicon nitride crystals are clearly visible.

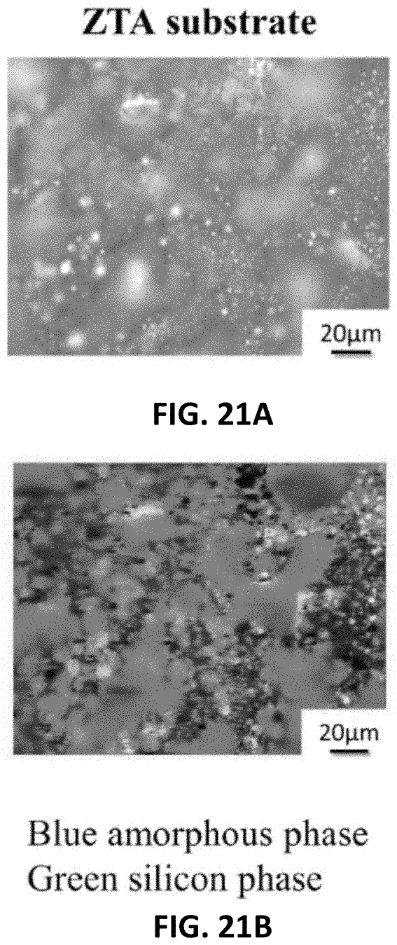

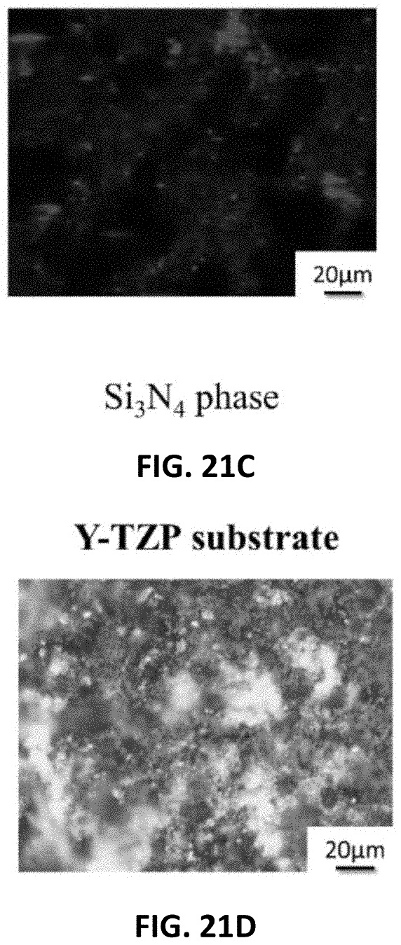

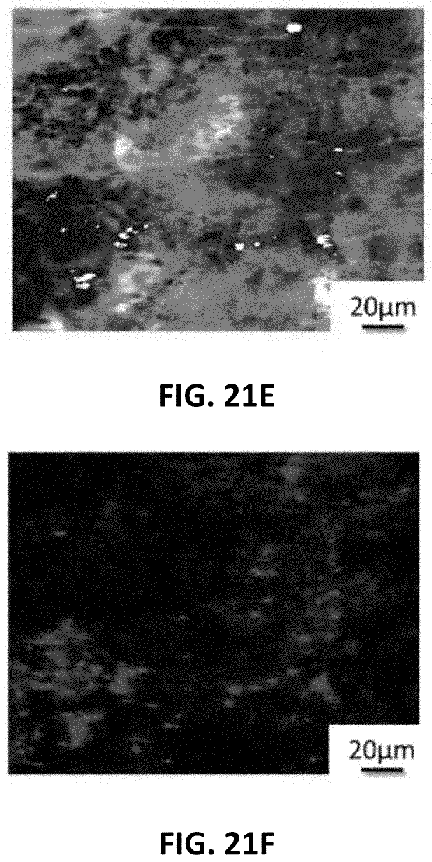

FIGS. 21A-21F shows the Raman imaging results obtained on the two ceramic substrates. For both materials, the blue/green compositional maps (FIGS. 21B and 21E) show amorphous and crystalline silicon signals, covering the entire surface. In the case of ZTA, the two phases are equally dispersed, with about a 60% amorphous signal, while for Y-TZP the crystalline phase is localized in a few spots, with a 80% amorphous phase. Both materials showed stoichiometrically-similar residual Si₃N₄ particle dispersion (FIGS. 21C and 21F).

Raman spectroscopic images obtained for the different substrates showed the appearance of a strong signal at about 520 cm⁻¹ related to the presence of Si—Si bonds formed due to the release of nitrogen from the surface. Depending on the position on the spectra, the Si—Si bonds may be associated with the presence of amorphous or nanocrystalline silicon, the latter being more abundant at higher power settings.

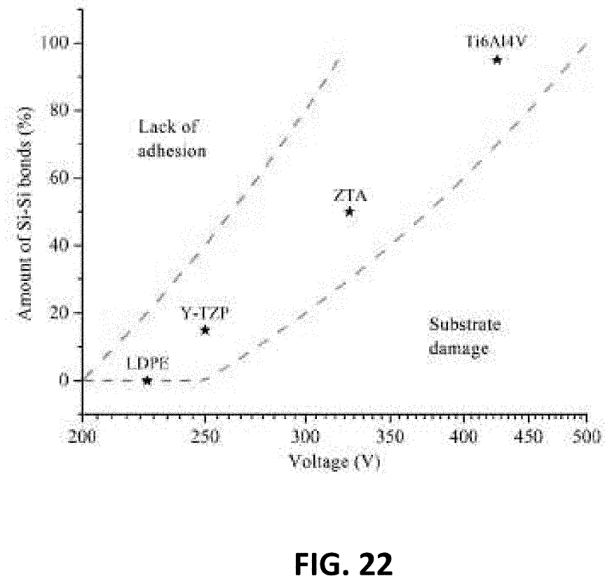

FIG. 22 shows a qualitative Si—Si bond dependency (measured by XPS), with respect to the applied power for each material. If the applied power is under a certain threshold, the coating does not adhere to the substrate and the powder is scattered by the laser beam. The threshold is dependent on the physical properties of the materials, such as melting point and absorbance coefficient. Cladding of the polymer was achieved at relatively low power levels, due to the melting of the matrix. This results in a virtually stoichiometric coating. In the case of ceramics, the coating required higher applied power to be deposited, but the stoichiometry of the coating changed into a silicon-rich phase. Although metals also require high power to attach the ceramic coating to the surface, this power melts the metallic substrate with the formation of a high content silicon-rich coating.

### Biological Testing

The antibacterial study pf the samples used gram-positive S. epidermidis. Staphylococcus epidermidis (14990®ATCCTM) cells were cultured in heart infusion (HI) broth at 37° C. for 18 h and titrated by colony-forming assay using brain heart infusion (BHI) agar. Aliquots of 1×10⁷ bacteria were diluted in 10 μL of phosphate-buffered saline (PBS) at physiological pH and ionic strength. The samples underwent preliminary UV sterilization and were distributed into wells. To each well, 1 mL of bacteria culture was added, and samples were incubated at 37° C. under aerobic conditions for 12, 24, and 48 h.

Cell viability was evaluated using a tetrazolium-based assay from a microbial viability assay kit (WST-8). Substrates with Staphylococcus epidermidis were collected at 24 and 48 h after cultivation and soaked in 1000 μL of PBS in 12-well plates. WST-8 solution was added to each well and Optical Density (OD) values were measured (the absorbance at 490 nm) using a plate reader EMax after incubation for 30-60 min.

SaOS-2 human osteosarcoma cells were used to test for osteoconductivity. They were first cultured and incubated in 4.5 g/L glucose DMEM (D-glucose, L-glutamine, phenol red, and sodium pyruvate) supplemented with 10% fetal bovine serum. They were allowed to proliferate within Petri dishes for about 24 h at 37° C. The final SaOS-2 concentration was 5×10⁵ cell/mL. The cultured cells were then deposited on the top surface of samples previously sterilized by exposure to UV-C light for 30 min. In osteoconductivity tests, cell seeding took place in an osteogenic medium which consisted of DMEM supplemented with about 50 pig/mL ascorbic acid, 10 mM 13-glycerol phosphate, 100 mM hydrocortisone, and ˜10% fetal bovine calf serum. The samples were incubated up to 14 days at 37° C. The medium was changed twice a week during the incubation period. Subsequently, the cells were stained for fluorescence microscopy with green dye to identify osteocalcin (Monoclonal, Clone 5-12H, dilution 1:500) and red dye to show osteopontin (Osteopontin, O-17, Rabbit IgG, 1:500).

After exposure to osteoblasts, each batch of samples was observed using fluorescence microscopy (BZ-X700). Prior to examination, the sample surfaces were treated with different immunostaining reagents, including Hoechst 33,342, anti-Human Osteocalcin Clone2H9F11F8, and Isotype IgG, Rabbit polyclonal antibody. Hoechst 33342, a cell nucleus stain (DAPI 4′,6-Diamidino-2-phenylindole, dihydrochloride, solution), served to visualize cell proliferation, while the other two antibodies were used to stain mineralization and bone matrix formation concentration quantifying the matrix proteins osteocalcin and osteopontin, respectively. Subsequently, a secondary antibody, Goat anti-Mouse IgG1 Antibody FITC Conjugated was added to enhance signal detection and visualization.

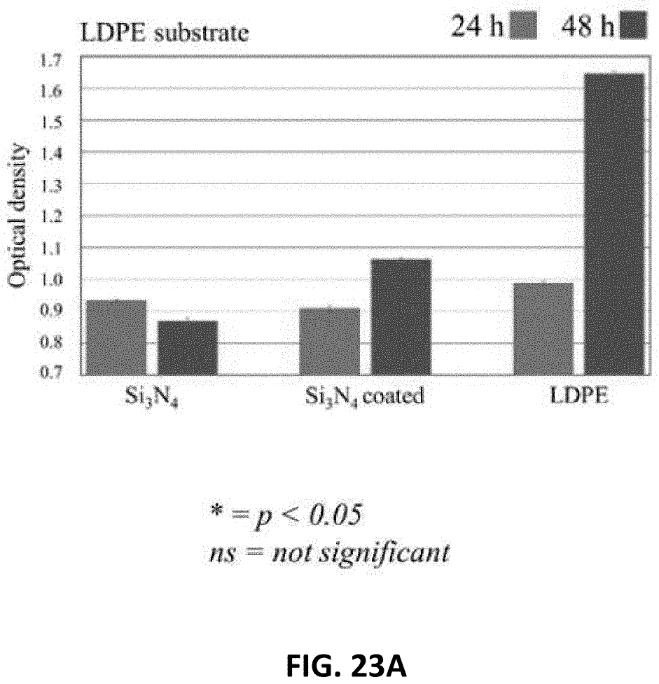

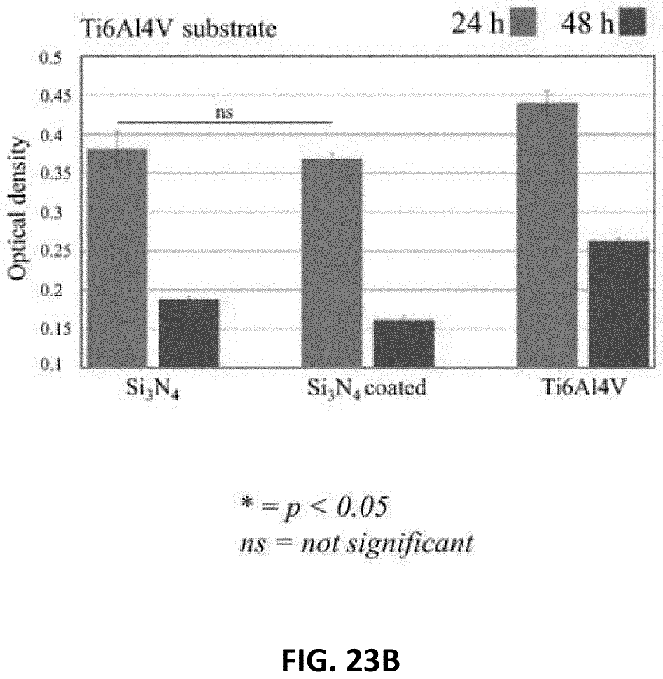

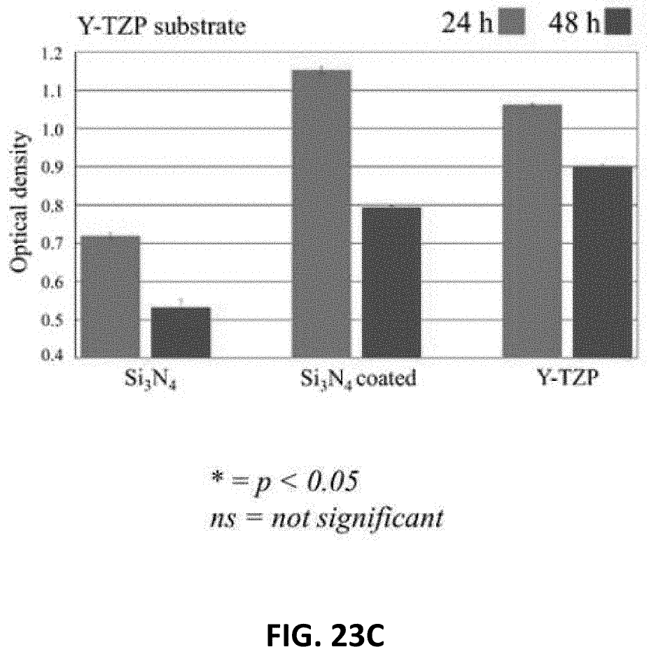

FIGS. 23A-23C show the microbial viability assays (WST) for three of the four substrates, compared with those of bulk silicon nitride and an untreated reference.

It was observed that Si₃N₄ reference samples usually displayed the lowest optical density at 48 h, with the exception of the Ti6Al4V substrate where its performance was comparable to that of the coated sample. The biological effects of silicon nitride were time-dependent and reach their maximum after about 12 h of treatment.

In the case of LDPE substrates, the trend observed for Si₃N₄ is the same, but on polymer and composite substrates, it changed completely. Si₃N₄ bulk is the only sample which showed that bacterial viability decreased over time. The LDPE substrates exhibit a growth of more than 60% in OD between 24 and 48 h possessing the highest bacterial amount in both the times. Despite the lowest OD value presenting at 24 h, the Si₃N₄-coated samples showed a slight variation, even if lower than that of the negative control.

In the case of Ti6Al4V, the microbial viability assay at 24 h showed a higher bacterial count on the Ti6Al4V uncoated sample than on identical uncoated or coated Si₃N₄ samples. At 48 h, a decreasing trend of OD was detected on all substrates. However, the Si₃N₄-coated substrate had the lowest OD value when compared with the two other bulk samples.

For zirconia samples (ZTA and Y-TZP show similar results), the silicon nitride coated samples treated with S. epidermidis had the highest 24-h living bacteria count. However, the lytic efficacy improved progressively with time. At 48 h, the optical density of the bacteria was 60% higher than on silicon nitride, but lower than on Y-TZP.

The fluorescence microscopy images obtained for various samples showed increased bone tissue production for the laser-cladded surfaces when compared to the uncoated substrates, as evidenced by both osteocalcin and osteopontin distributions.

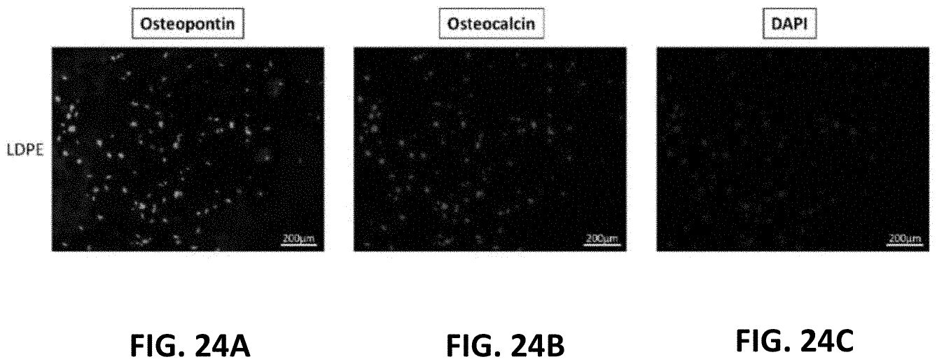

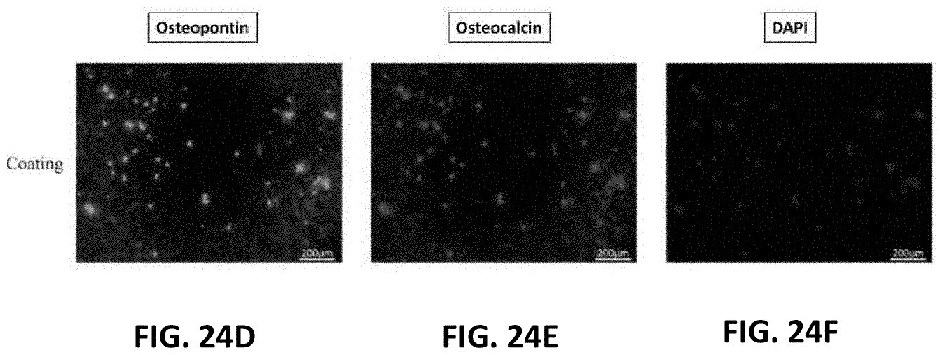

FIGS. 24A-24F show the results of the fluorescence microscopy testing with markers for nuclei (blue), osteocalcin (green), and osteopontin (red) for an LDPE substrate (FIGS. 24A-24C) and a silicon nitride coated LDPE sample (FIGS. 24D-24F). Unlike zirconia and Ti6Al4V, the uncoated LDPE substrates appear to be colonized by cells and coating application does not significantly affect the amount or distribution of either cell nuclei or bone tissue.

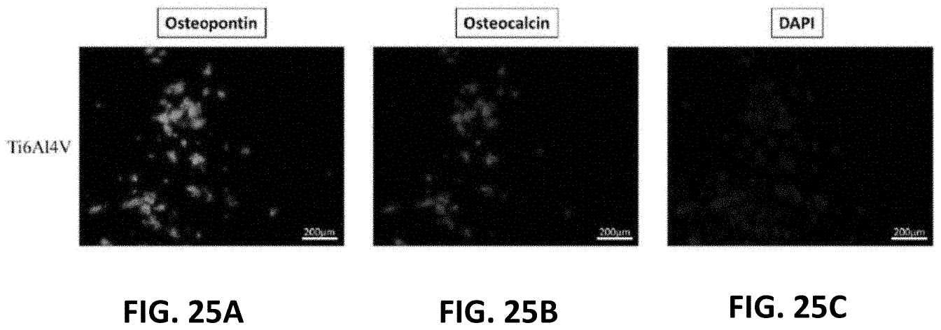

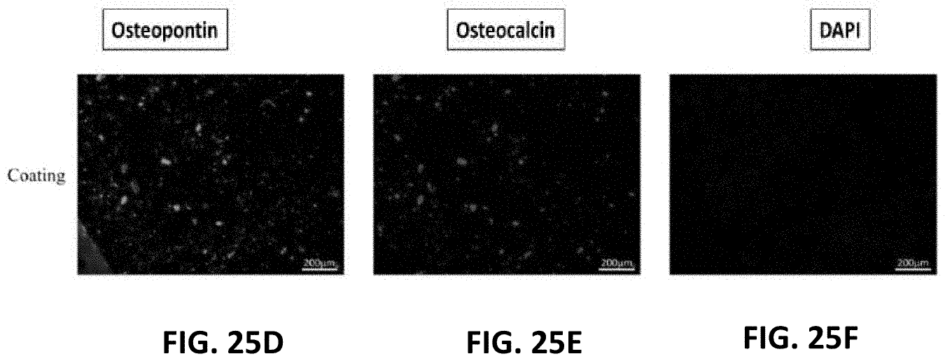

FIGS. 25A-25F show the results of the fluorescence microscopy testing with markers for nuclei (blue), osteocalcin (green), and osteopontin (red) for a Ti6Al4V alloy substrate (FIGS. 25A-25C) and a silicon nitride coated Ti6Al4V sample (FIGS. 25D-25F). It was observed that cell nuclei are not homogeneously distributed on the sample surface, forming agglomerates at the center where bone tissue (osteopontin and osteocalcin) also formed. Coated samples, on the other hand, were homogeneously covered.

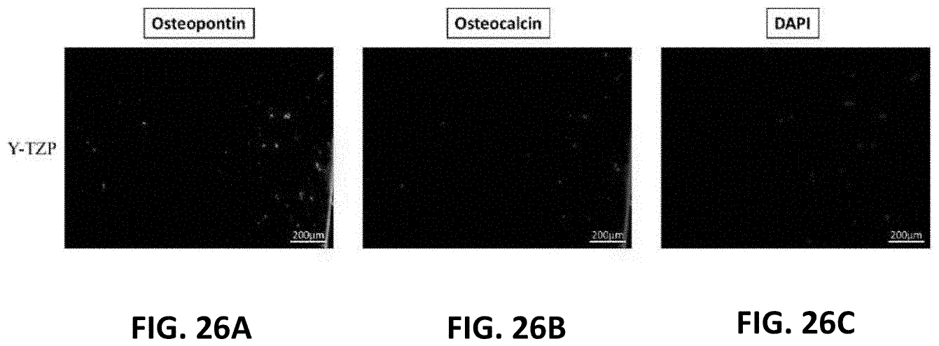

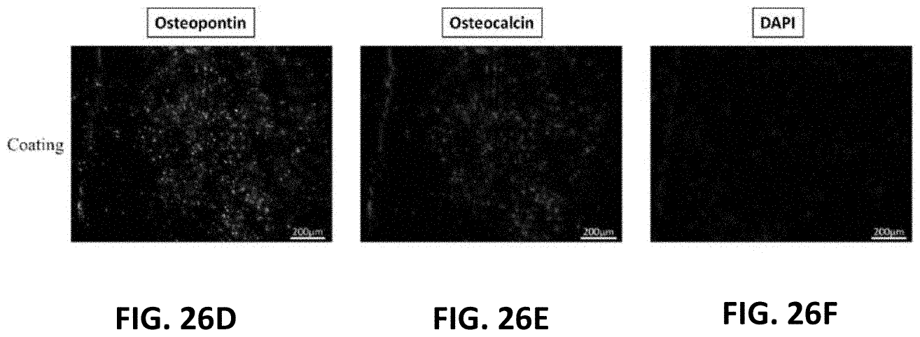

FIGS. 26A-26F show the results of the fluorescence microscopy testing with markers for nuclei (blue), osteocalcin (green), and osteopontin (red) for a Y-TZP substrate (FIGS. 26A-26C) and a silicon nitride coated Y-TZP sample (FIGS. 26D-26F). ZTA and Y-TZP show similar results. On the uncoated substrate, three signals for the non-collagenous bone matrix proteins in osteoblasts were observed: osteocalcin, osteopontin, and for cell nuclei. All were barely visible and only localized in a few spots. Moreover, the increased distribution of blue DAPI signals indicates that more nuclei than bone tissue formed. When the coating was applied, osteopontin and osteocalcin were strengthened and more uniformly distributed over the entire area.

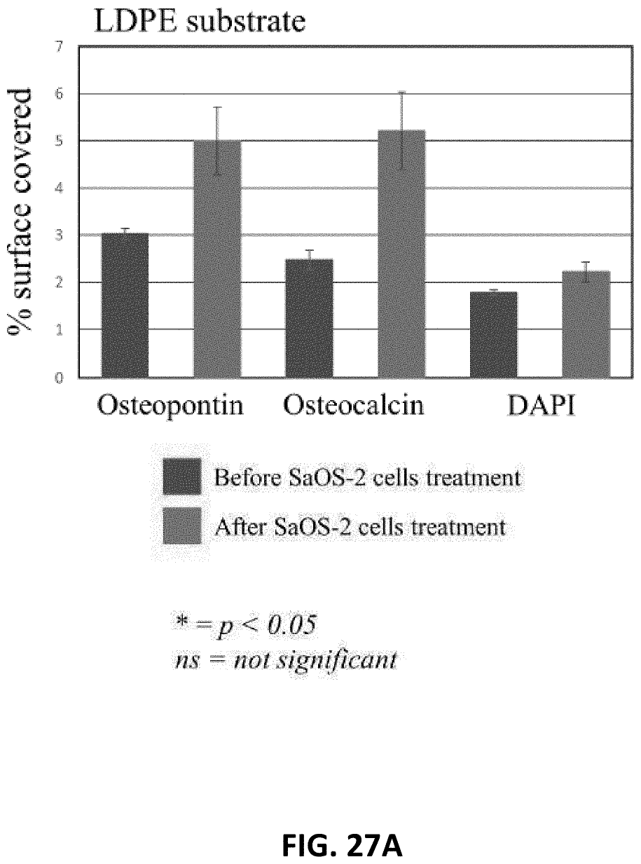

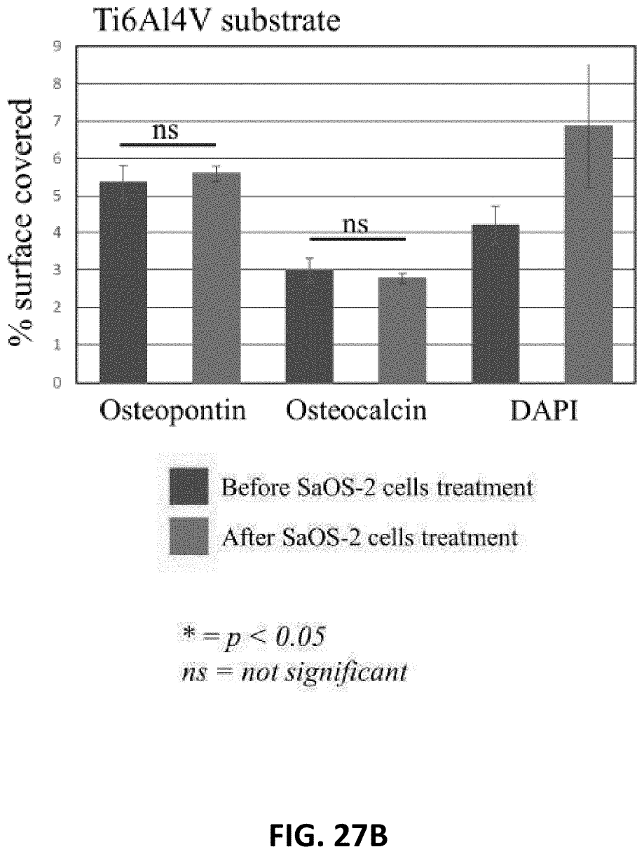

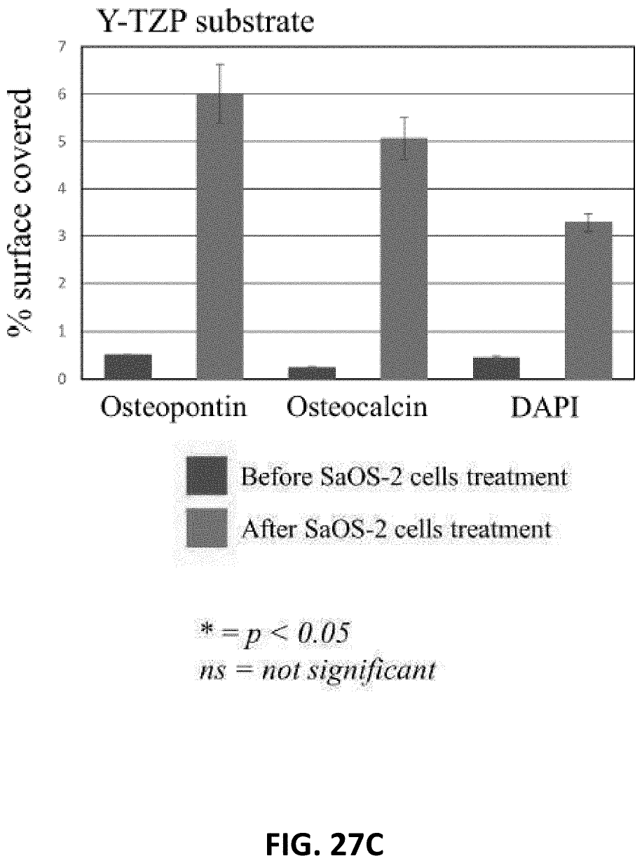

FIGS. 27A-27C show the fluorescence analysis of substrate surface bone and cell nuclei before and after treatment. For the LDPE substrate, the bone tissue increased around 2%, but the percentage of cell nuclei still present on the surface was nearly identical to that of the untreated substrate. For the Ti6Al4V, the number of nuclei was higher than that of the untreated samples, but bone formation was virtually the same for both samples. For the Y-TZP substrate, the histogram confirmed a higher amount of surface bone tissue formation. An increase of 5-6% of the bone product and 3% of nuclei on the coated surface was evident.

The in vitro bacteria counts summarized in FIGS. 27A-27C confirmed that all silicon nitride-based cladded layers have time-dependent antibacterial effects. The trend observed in Y-TZP- and Ti6Al4V-coated substrates showed a clear decrease over time. In the LDPE Si₃N₄-coated substrates, the opposite trend was observed, but the increase was less significant compared with that of uncoated LDPE. The slight increase of OD at 48 h may be associated with adopted laser treatment because, although particles were fixed without physically modifying the bulk substrates of Ti6Al4V and Y-TZP, the surface was melted by the laser. In LDPE, this resulted in Si₃N₄ particle incorporation. This provided a non-homogeneously Si₃N₄ covered surface, but areas with LDPE were still present as observed in FIGS. 18A-18B.

More bone tissue was formed on substrates with higher fractions of surface silicon phase. In silicon-rich samples, cells produced bone tissue, but at a lower rate than that of other materials.

Substrate roughness also contributed actively to bone formation. The ZTA and Ti6Al4V samples achieved higher surface roughness after laser cladding when compared to other substrates. This influenced the biological response.

### Example 3

### Laser-Cladding Manufacturing Process to Deposit a Si₃N₄ Coating onto Porous Ti-Alloy

Si₃N₄-Coating Fabrication and Characterizations

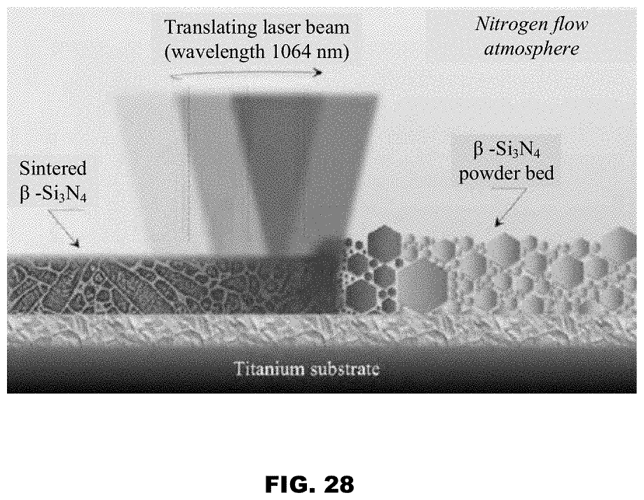

The starting Si₃N₄ powder used for the laser-cladded Si₃N₄ coating consisted of 90 wt. % of β-Si₃N₄ which had previously been reacted with 6 wt. % yttrium oxide (Y₂O₃, Grade C) and 4 wt. % aluminum oxide (Al₂O₃, SA8-DBM). The resulting powder formed a β-SiYAlON structure. A Vision LWI VERGO-Workstation equipped with a Nd:YAG laser with a wavelength of 1064 nm (max pulse energy: 70 J, peak power 17 kW, voltage range 160-500 V, pulse time 1-20 ms, spot size 250-2000 μm) was used to achieve densification of successive layers of Si₃N₄ powder placed on the surface of the titanium substrate. The apparatus operated under a constant flux of nitrogen gas to limit Si₃N₄ decomposition and oxidation. The Nd:YAG laser was operated with a spot size of 2 mm, driven by an applied voltage of 400 V, and a pulse time of 4 ms. The operation was repeated until a continuous coating thickness of t=15±5 μm was obtained over the entire surface of the Titanium substrate. Square 1 cm×1 cm (0.5 cm thickness) samples were used for all the experiments. The fabrication procedure of the Si₃N₄ coating is schematically depicted in FIG. 28.

Cross-section crystallographic analyses were performed into a scanning electron microscope (SEM) equipped with an Electron-Backscattered X-ray Diffraction (EBSD) detector. The microhardness was then tested using a DUH-211S dynamic ultra-micro hardness tester (Shimadzu) with a triangular diamond tip and maximum applied force of 60 mN.

Scratch testing was performed on coated titanium substrates according to the ASTM C1624-05 Standard (reapproved in 2010) using a diamond stylus with a conical Rockwell geometry (included angle 120 degrees and tip radius equal to 200 μm). A progressively increasing vertical force was applied from 0.5 up to 25 N.

The same procedure used for coating Ti-alloy substrates was applied to coat in toto commercially available acetabular shells (G7® OsseoTi Limited); uncoated components served as controls (n=3 per group). The hip components were made of Ti-alloy and possessed a porous surface morphology appropriated for cementless socket fixation to the pelvis (i.e., with a pore size in the order of 1 mm).

The surface morphology of the porous Ti-alloy acetabular shells before and after laser Si₃N₄-coating was characterized with a confocal scanning laser microscope capable of high-resolution optical images with depth selectivity. All images were collected using 20× magnification. Scanning Electron Microscopy (SEM) and Energy Dispersive X-ray Spectroscopy (EDS) (JSM-700 1F) were used to acquire high resolution images and chemical composition maps of both cross-sections and top-surface of Si₃N₄-coated substrates after scratch testing. Cross-sections were obtained by diamond-blade cutting and successive fine polishing with diamond paste.

### Osteoconductivity Test

SaOS-2 human osteosarcoma cells were cultured and incubated in 4.5 g/L glucose DMEM (D-glucose, L-Glutamine, Phenol Red, and Sodium Pyruvate) supplemented with 10% fetal bovine serum. The cells were then allowed to proliferate within petri dishes for about 24 hours at 37° C. After adjusting the final cell concentration at 5×10⁵ cell/ml, the cultured cells were deposited on the surface of Si₃N₄-coated and uncoated Titanium substrates (n=3 each type) previously sterilized by exposure to UV light. In osteoconductivity tests, cell seeding was performed in an osteogenic medium, which consisted of DMEM supplemented with about 50 μg/mL ascorbic acid, 10 mM 8-glycerol phosphate, 100 mM hydrocortisone, and 10% fetal bovine calf serum. The samples were incubated up to 7 days at 37° C. The medium was changed twice during the week of incubation. Immunocytostaining was performed as follows. SaOS-2 were fixed with 4% paraformaldehyde for 15 minutes followed by 30 minutes incubation at room temperature with the following primary antibodies: mouse antihuman Gla osteocalcin and rabbit anti-human osteopontin (dilution=1:500). The cells were then incubated with fluorescence conjugated and secondary antibodies Goat anti-Mouse Antibody FITC Conjugated and Goat anti-Rabbit Antibody PE Conjugated and (1:200). Cell nuclei were stained with stained with Hoechst 33342 (1:100). The staining was observed under a fluorescent microscope. Confocal scanning laser microscopy images were obtained with a 3D laser confocal microscope (OLS4000-SAT; Olympus Co.) in order to assess the volumetric amounts per unit area of bony hydroxyapatite (Hap) produced by the SaOS-2 cells in one week. The amounts of osteocalcin and osteopontin in the deposited bony tissue were also estimated by direct pixel counting on fluorescence micrographs using automatic software.

### Antibacterial Tests

Freeze-dried pellets of Staphylococcus epidermidis (ATCCTM 14990®) (S. epidermidis, henceforth) were hydrated in heart infusion (HI) broth and incubated at 37° C. for 18 hours in brain heart infusion (BHI) agar. The mixture was subsequently assayed for colony forming units (CFU) and diluted to a concentration of 1×10⁸ CFU·mL⁻¹ using phosphate-buffered saline (PBS). An aliquot of 100 μL of the bacterial suspension was spread onto individual BHI agar plates. The Si₃N₄ substrate samples (previously UV-sterilized) were then placed in contact with the agar for inoculation purposes, followed by incubation at 37° C. under aerobic conditions for 24 and 48 hours.

Cell viability was evaluated by a tetrazolium-based assay using the Microbial Viability Assay Kit (WST-8). 24 and 48 hours after cultivation, substrates with Staphylococcus epidermidis were collected and soaked in 1000 μL of PBS in 12-well plates. WST-8 solution was added to each wells and OD values measured (the absorbance at 490 nm) using plate reader EMax after incubation for 30-60 minutes.

At each time point, the test and control samples were observed by fluorescence microscopy. For visualization, bacteria were stained with two different solutions: (i) 4′,6-diamidino-2-phenylindole (DAPI) which binds to and stains DNA blue thereby imaging the nucleus location; (ii) 5 (6)-carboxyfluorescein diacetate (CFDA); and, (iii) propidium iodide (PI). PI's red color highlighted dead or injured bacteria. Conversely, CFDA's green color revealed living bacteria. The staining protocol consisted of adding 1 μL of DAPI, the PI solution, and 15 μL of CFDA solution to the samples, and then incubating them for 5 minutes at 37° C. After removing the buffer, the cells were analyzed under the fluorescence microscope. Quantitative assessments of the presence of living bacteria on the Si₃N₄ substrates were then directly obtained from automatic image analyses of the above micrographs by using >12 different fluorescence micrographs for each sample and n=3 samples for each exposure-time condition.

Escherichia coli (25922° ATCCTM) (E. coli, henceforth) was cultured at Kyoto Prefectural University of Medicine and the Dr. GENE 4: E. coli Transformation Kit was used to transform the vector (firefly luciferase gene) into E. coli cells. Previously sterilized by UV, Si₃N₄-coated and uncoated Ti-alloy acetabular shells (n=3 each) were spread with the bacterial solution (1×10⁸ CFU/mL) in brain heart infusion (BHI) agar at 37° C. Incubation at 37° C. under aerobic conditions lasted for 48 hours and the shell samples were successively subjected to luciferase firefly gene transformation. For doing so, a solution containing luciferin was added on the top of the acetabular shell samples. Upon reaction among adenosine triphosphate, Mg2+, O2, and luciferase enzyme, a “glowing” chemiluminescent signal could be captured by means of a CCD camera on the acetabular shell in toto, which revealed the presence of living bacteria.

### Statistical Analyses

Data relating to osteogenesis and antibacterial properties were analyzed by calculating their mean value±one standard deviation. The Student's t-test was used to detect statistically significant differences between data, p values <0.01 being considered statistically significant and labeled with two asterisks in the figures.

Structural, Chemical, and Mechanical Characteristics of Si₃N₄ Coating

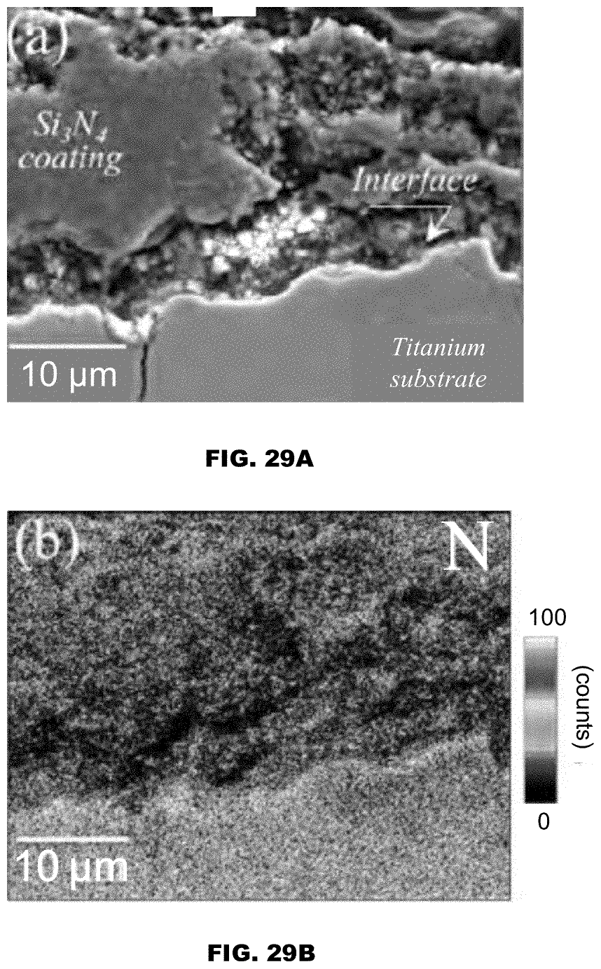

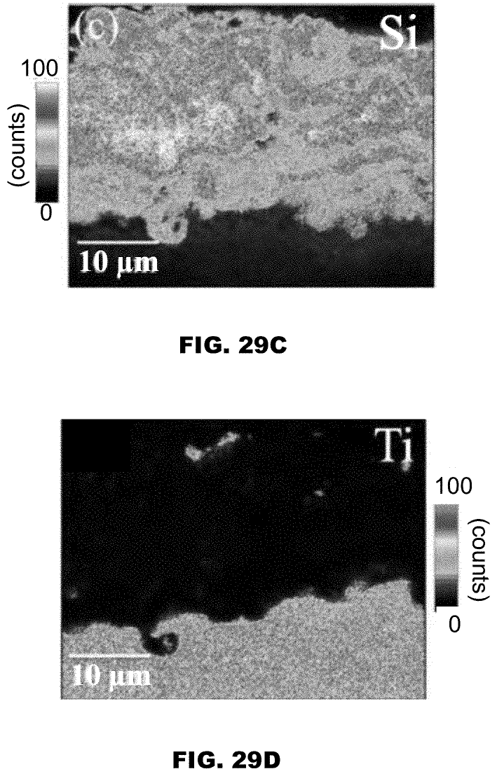

FIGS. 29A-29D show SEM/EDS analyses performed on the cross-section of a Si₃N₄-coated Ti-alloy substrate. The average thickness of the coating was t=15±5 μm. The main feature in the SEM micrograph (FIG. 29A) is the wavy morphology of the ceramic/metal interface, which showed clear interlocking features between metal and ceramic phases. Some residual porosity could have been retained in the coating, although it was not directly observed by optical or scanning electron microscopy. The rough surface morphology was due to the inhomogeneous size of the silicon nitride powder (5±50 μm). EDS elemental analyses of N, Si, and Ti (in FIGS. 29B, 29C, and 29D, respectively) revealed the presence of Nitrogen on both coating and substrate, Si-rich areas in coating area and no diffusional transport of the Ti element into the coating during laser-cladding (the very weak Ti contamination in the coating area is believed to be an artifact of the cutting/polishing procedure applied to obtain the cross section).The low detectability of Nitrogen and the surface morphology of the coating made it impossible to quantify the amount of Nitrogen diffused in the substrate and the amount that was retained by the coating. However, it was clear that the treatment did not produce a TiN layer. The absence of Ti in the chemical composition of the coating shows that partial melting of the metallic alloy under the laser beam took place only in the immediate vicinity of its surface (and was responsible for the wavy interface morphology). A small relatively intense Titanium signal was observed on the top of the coating in FIG. 29D. This was caused by splashes of molten titanium that occurred when the laser hit portions of substrate not completely covered by ceramic powder.

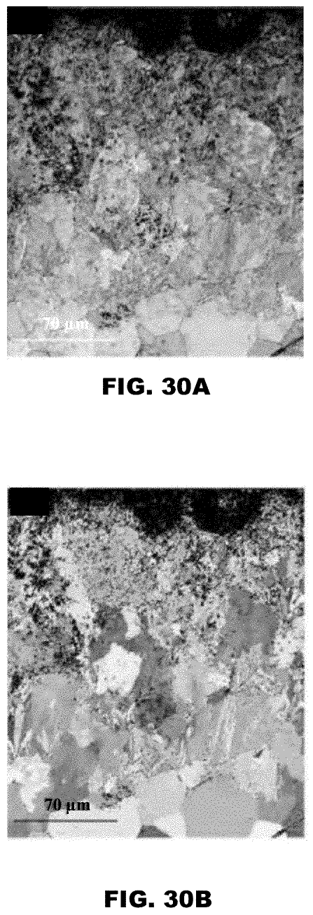

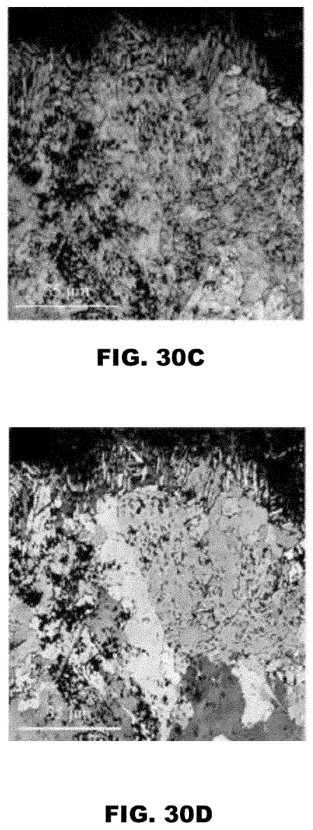

FIGS. 30A-30D show the EBSD images of the cross section of the titanium samples coated with silicon nitride. Low magnification images, in FIGS. 30A and 30B show the grain structure and the orientation of the α-titanium phase to a depth of about 200 μm. During laser treatment, the titanium grains were affected and distorted by the heat treatment for the first 100 μm in depth, with the formation of crystal twinning, in particular in the region between 100 and 150 μm. Beyond 150 μm, the microstructure was unaffected by the laser. FIGS. 30C and 30D show an area close to the Si₃N₄ interface at higher magnification. The titanium grains have an acicular shape with a preferential orientation orthogonal to the interface itself. The acicular region is followed by an area of elongated grains with a size comparable to the unaffected areas of FIGS. 30A and 30B The black spot present on the images are intermetallic phases of titanium and silicon, which could not be identified by EBSD because of their wide range of stoichiometry.

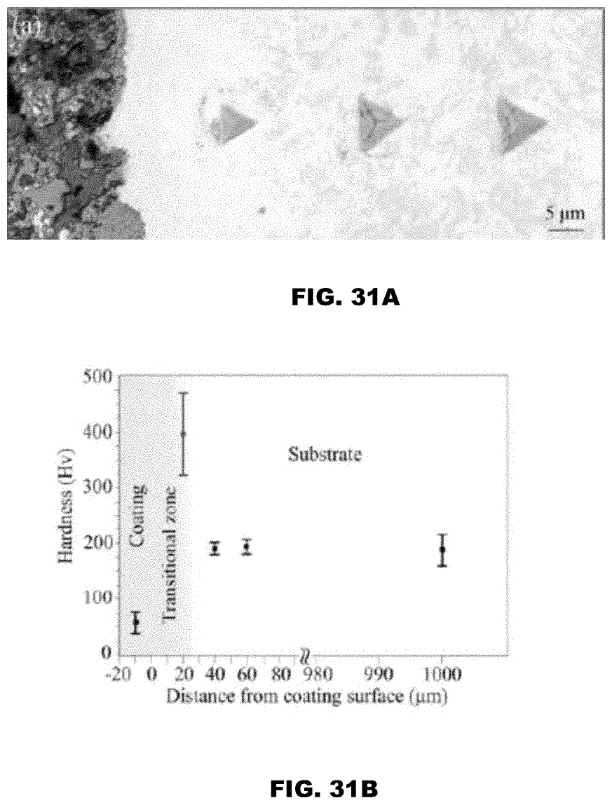

FIGS. 31A and 31B show the micro hardness results obtained on a cross section of the sample. FIG. 31A shows three indentations performed at different distances from the interface. It was observed that the first indentation on the left was smaller when compared with the others, which have a similar size. FIG. 31B shows the average microhardness of the sample in the coating as a function of distance from the interface between coating and substrate. The microhardness of the coating could not be measured on the top surface due to roughness and inhomogeneities. The average value shown in the plot of FIG. 31B refers to measurements on the cross section. On the Titanium substrate, the hardness near the coated surface was higher compared to the bulk material. This is due to a heating effect during the laser-fabrication of the coating. Local heating produced smaller grains which locally increased the hardness of the material.

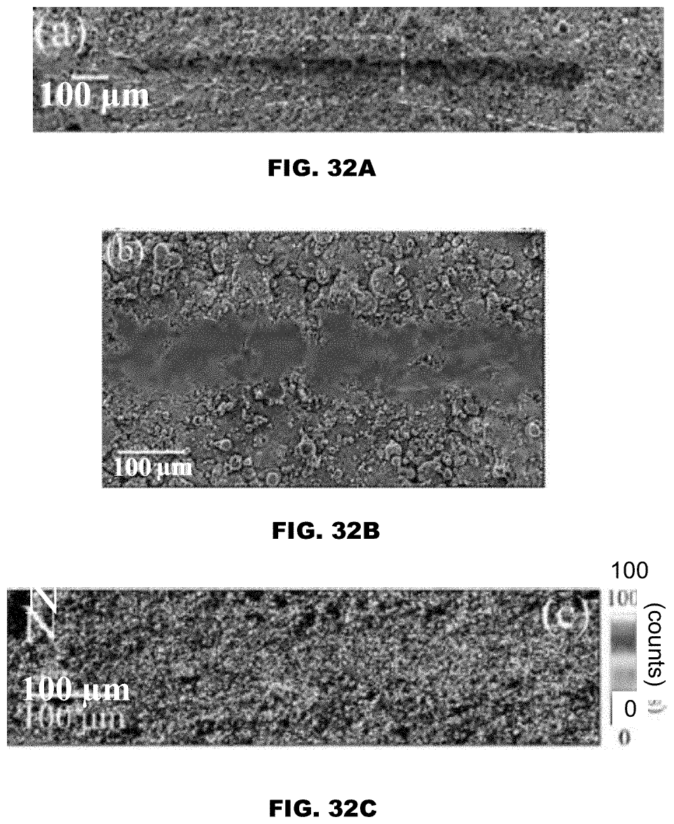

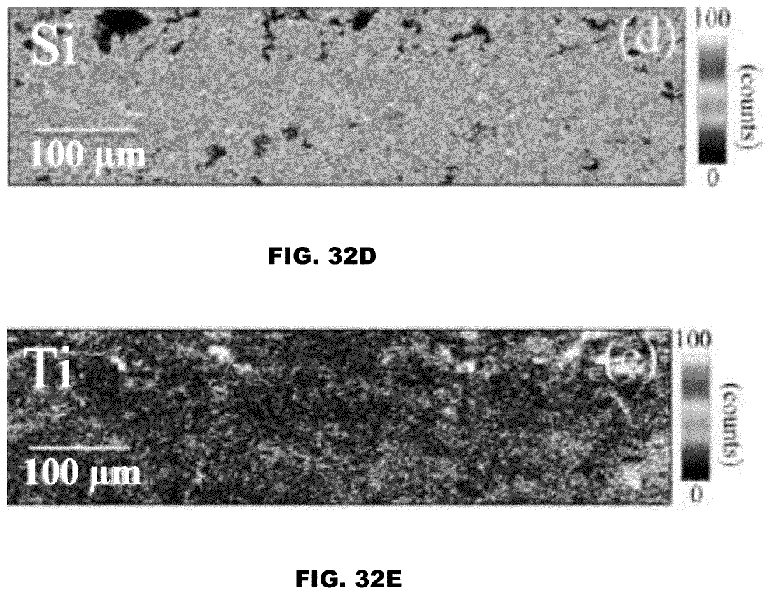

FIGS. 32A and 32B show low- and high-magnification SEM micrographs, respectively, taken on the top surface of the Si₃N₄ coating at the location where scratch testing was applied (progressively from left to right) with a load from 0 up to 25 N for a length about 1 mm. The scratch exerted by the conical diamond stylus introduced plastic deformation on the surface of Si₃N₄ coating. However, neither microcracks nor areas of spalling in the coating were observed around the scratch, which demonstrates the high interfacial strength of the coating to the alloy substrate. Elemental EDS analyses in FIGS. 32C, 32D, and 32E show the chemical distributions at the scratch site for N, Si, and Ti elements, respectively. These EDS data indicated no Ti contamination inside the scratched area, thus demonstrating that the running stylus did not reach the Ti-alloy layer during testing. However, some local spots of Titanium were seen in the images on the top surface of the coating due to splashing of molten metal during laser treatment in areas not completely covered by Si₃N₄ powder.

Osteoconductivity of Si₃N₄ Coating

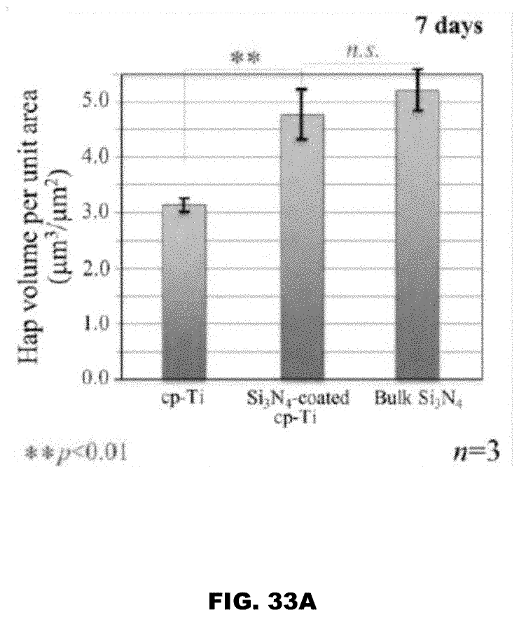

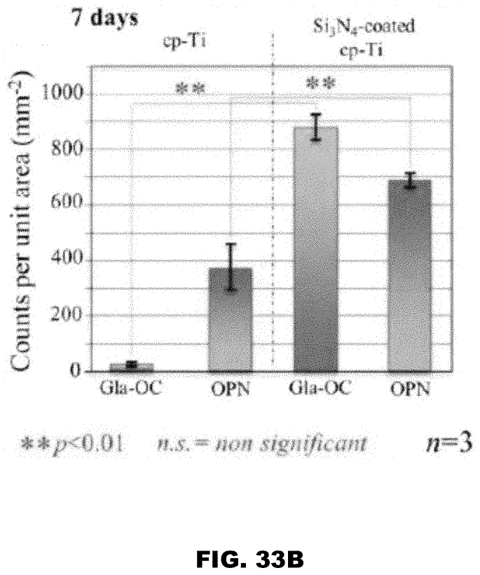

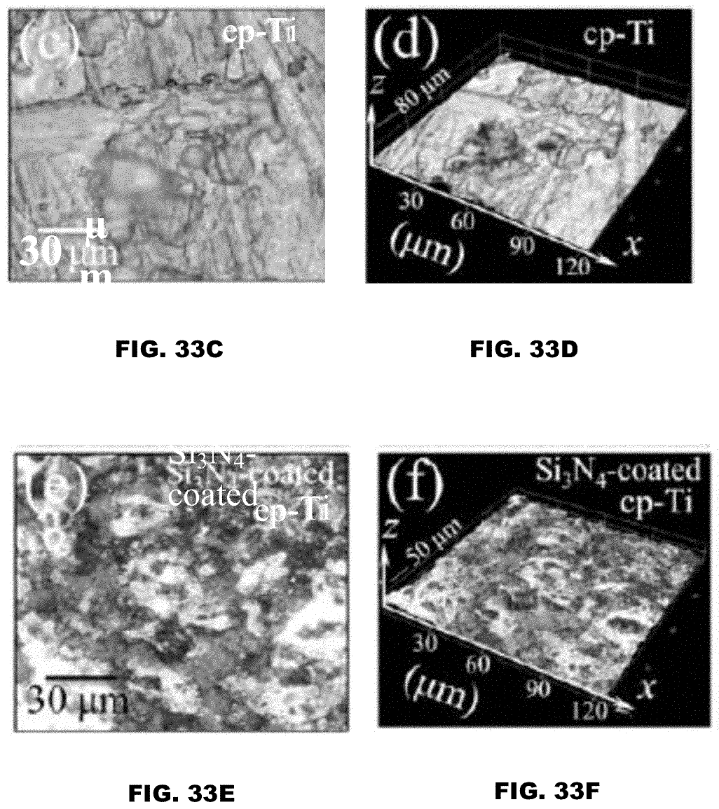

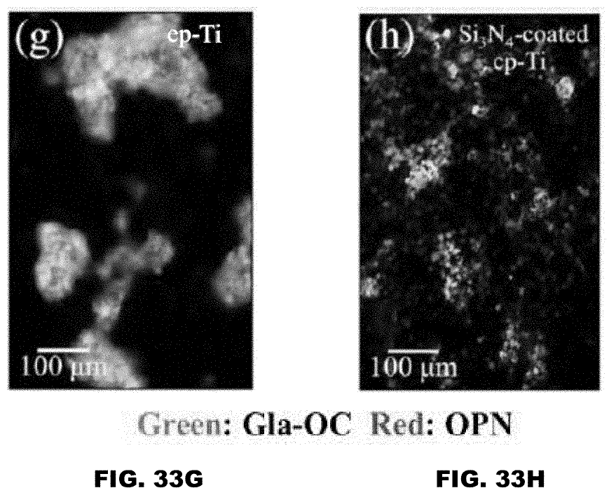

FIG. 33A compares the volumetric amounts of grown bone tissue per unit area by SaOS-2 cells during 7 days of incubation as measured by laser microscopy on uncoated and Si₃N₄-coated Ti6Al4V substrates, in comparison with bulk Si₃N₄. In section (b) of the same figure, the composition of the bone tissue was evaluated with respect to its Gla-osteocalcin and osteopontin contents. Laser microscopy images for the uncoated alloy (FIGS. 33C and 33D) and Si₃N₄-coated alloy (FIGS. 33E and 33F) substrates are shown in planar and three-dimensional projections. A homogeneous layer of bony apatite formed on the Si₃N₄-coated substrate, with a maximum height of ˜50 μm. The total amount of hydroxyapatite (calculated over the entire substrate's surface) showed no statistically meaningful difference for bulk Si₃N₄ under the same testing conditions. Conversely, the amount of bone tissue grown on the Si₃N₄-coated Ti-alloy substrate was ˜50% higher than that deposited by cells on the uncoated Ti-alloy substrate (cf. FIG. 33A). The SaOS-2 cells grew tissue only in very localized microscopic areas (cf. FIGS. 33C and 33D), in which the height of the deposited apatite reached up to ˜80 μm.

The fractions of Gla-osteocalcin and osteopontin (given as counts per unit area) greatly differed between coated and uncoated substrates. FIG. 33B. FIGS. 33G and 6H show merged fluorescence images of Gla-osteocalcin (green) and osteopontin (red) for bone tissue grown on uncoated and Si₃N₄-coated Ti-alloy substrates, respectively. These data show that the bone tissue grown by cells on the uncoated Ti-alloy had an unbalanced composition rich in osteopontin. Conversely, the bone tissue deposited on the Si₃N₄-coated Ti-alloy was richer in Gla-osteocalcin, with an osteocalcin/osteopontin balance resembling that of natural bone. This consistency between laser microscopy and biological assay data, shown in FIGS. 33A and 33B, substantiated the effect of the Si₃N₄ coating in enhancing the osteogenic efficiency of human osteoblasts.

Antibacterial Properties of Si₃N₄ Coating

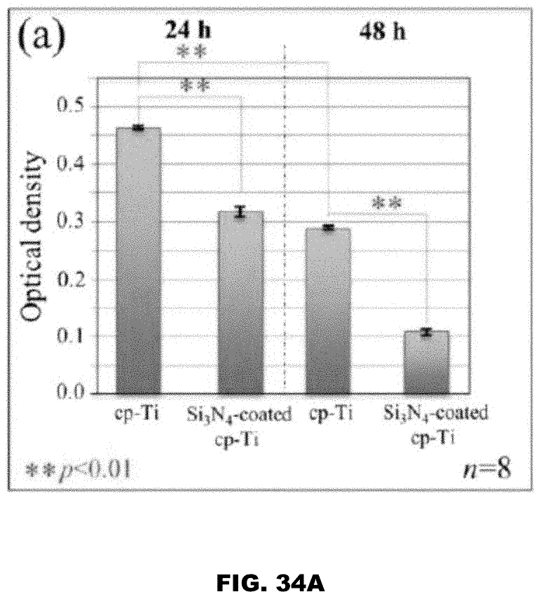

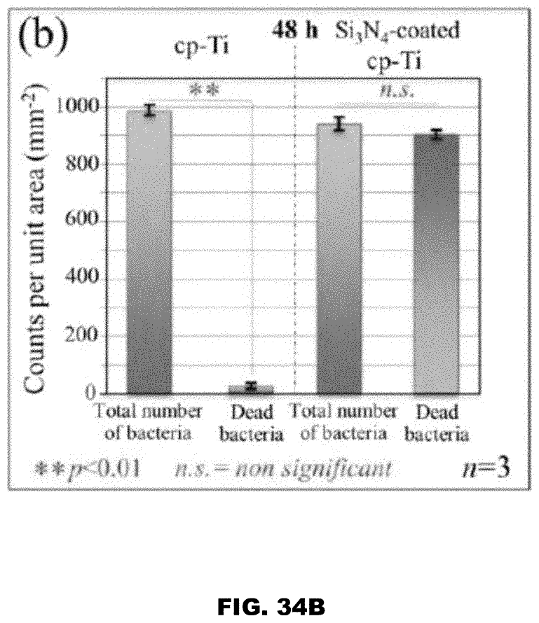

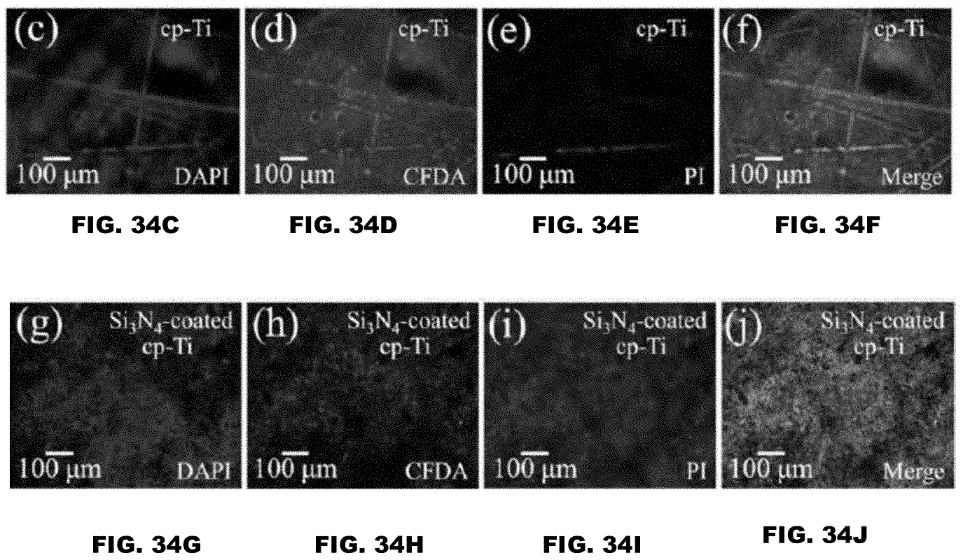

FIGS. 34A and 34B show data representing the metabolic responses of Gram-positive S. epidermidis after exposure to uncoated and Si₃N₄-coated Ti6Al4V-alloy substrates in terms of optical density and fraction of dead bacteria, respectively. Cell viability data in FIG. 34A, which at the exposure times of 24 and 48 hours, showed the existence of a larger population of bacteria on the uncoated substrate compared to the Si₃N₄-coated substrate. A decreasing trend over time for the bacterial population was detected on both substrates. However, the rate was clearly higher for the Si₃N₄-coated substrate. Moreover, the fraction of dead bacteria at 48 hours was negligible on the uncoated substrates, while nearly all bacteria underwent lysis on the Si₃N₄-coated substrates (FIG. 34B). DAPI (blue), CFDA (green), PI (red), and DAPI/CFDA/PI-merged fluorescence micrographs (taken at 48 hours) clearly show the antibacterial performances of uncoated and Si₃N₄-coated substrates (in FIGS. 34C-34F and 34G-34J respectively). Optical density data (in FIG. 34E) provide direct assessments of the efficacy of the Si₃N₄ coating in reducing bacterial adhesion. The significant difference seen in the density of red pixels (cf. FIGS. 34E and 34I for uncoated and Si₃N₄-coated substrates, respectively) suggests that the Si₃N₄ coating induced lysis in Gram-positive S. epidermidis. Note that some antibacterial effect (although limited to a reduction in bacterial adhesion) was also found for the uncoated Ti-alloy sample. This effect is well-known and has been attributed to the presence of a TiO₂ passivation layer at the surface of the alloy.

Application of the Si₃N₄-Coating Method to Commercially Available Acetabular Shells

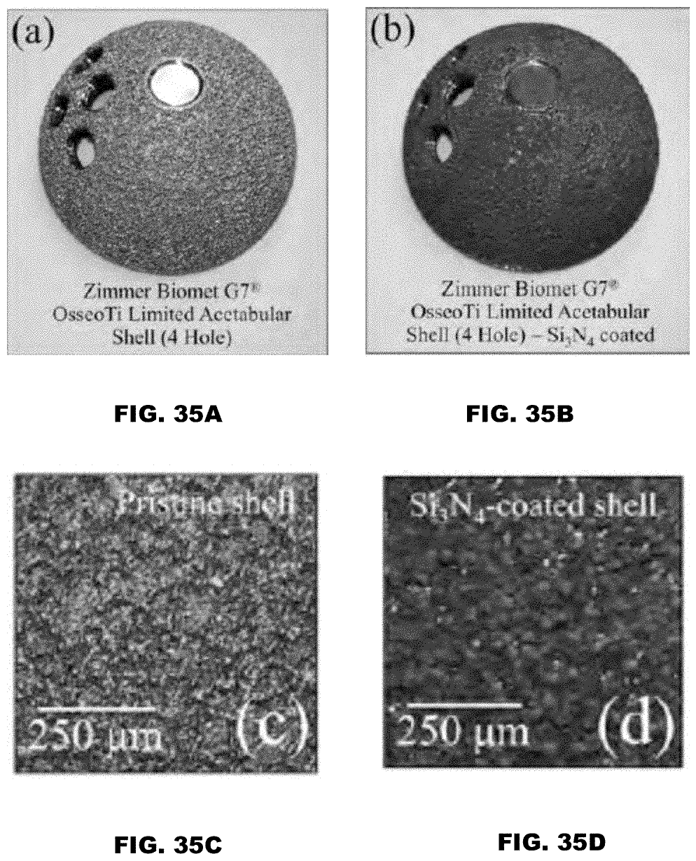

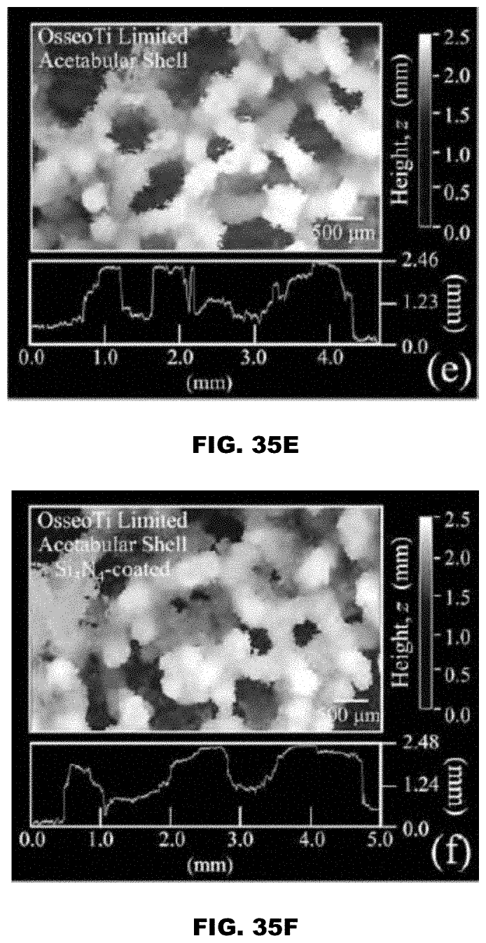

To show the practical feasibility of Si₃N₄ laser-deposition technology, it was applied it to commercial porous Ti-alloy acetabular shells. The shells were designed for cementless fixation to the human pelvis during total hip replacement. Shells were coated with Si₃N₄ on their external porous convex surface which comes into direct contact with host bone upon implantation. FIGS. 35A and 35B show an uncoated and a Si₃N₄-coated acetabular shell, respectively. The coating procedure and the laser-cladding conditions were the identical to those described in the previous sections. The microstructures of uncoated and Si₃N₄-coated acetabular shells are shown in the high-resolution optical images of FIGS. 35C and 35D, respectively. The results of quantitative laser microscopy assessments on the structure of the porous shell before and after the Si₃N₄-coating procedure are shown in FIGS. 35E and 35F, respectively. Critically, from a commercial standpoint, and, as shown in these two figures, the Ti-alloy surface preserved its porous morphology even after deposition of the Si₃N₄ coating.

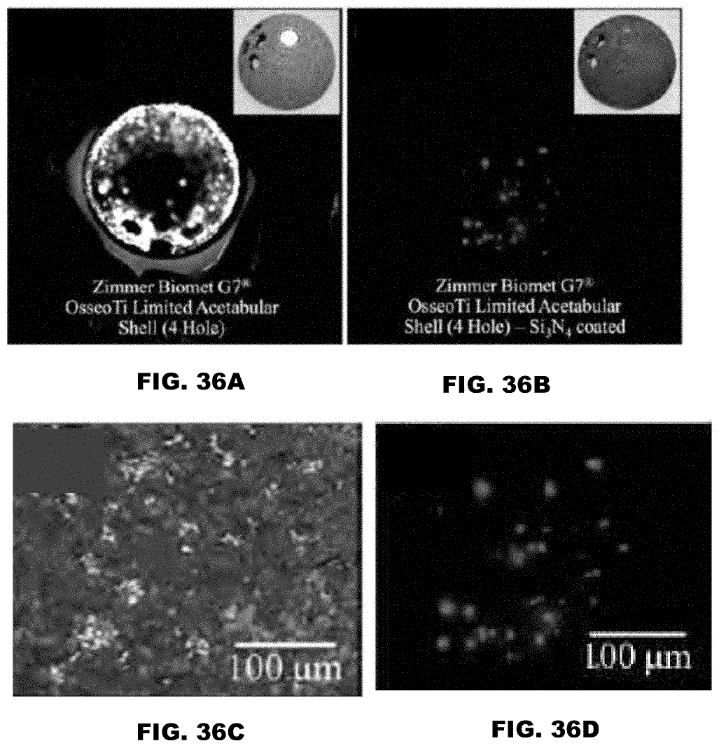

FIGS. 36A and 36B show the results of luciferin “glowing” experiments on Gram-negative E. coli for uncoated and Si₃N₄-coated Ti-alloy acetabular shells, respectively. The chemiluminescent oxyluciferin luciferase signal shows the degree of bacterial contamination between the acetabular shells in toto: the uncoated shell was bright with fluorescent bacteria, while the coated shell was essentially dark with a limited number of spots. These spots corresponded to microscopic areas in which the coating was not completely covering the porosity.

### The Common Origin of Osteogenic and Antibacterial Properties

Reactive nitrogen species (RNS) such as N₂O, NO, and ⁻OONO are highly effective biocidal agents. Spontaneous RNS elution from Si₃N₄ may discourage surface bacterial adhesion and activity. Unlike other direct eluting sources of exogenous NO, Si₃N₄ elutes NH₄⁺ and a small fraction of NH₃ at physiological pH due to surface hydrolysis and homolytic cleavage of Si—N covalent bonds. Ammonium NH₄⁺ can enter the cytoplasmic space of cells in controlled concentrations and through specific transporters. This ion is a nutrient used by cells to synthesize proteins for enzymes and genetic compounds. Together with the leaching of orthosilicic acid and related compounds, NH₄⁺ upregulates osteoblastic activity, and stimulates collagen type 1 synthesis. Conversely, ammonia NH₃ can freely penetrate the external membrane and target the stability of DNA/RNA structures in bacterial cells. However, the release of unpaired electrons from the mitochondria in eukaryotic cells activates a cascade of consecutive reactions, which starts with NH₃ oxidation into hydroxylamine NH₂OH (ammonia monooxygenase) along with an additional reductant contribution leading to further oxidation into NO₂ through a process of hydroxylamine oxidoreductase. This process involves nitric oxide NO formation. The elution kinetics of nitrogen is slow but continuous, thus providing long-term efficacy against bacterial colonies including mutations. However, when slowly delivered, NO radicals have been shown to act in an efficient signaling pathway leading to enhanced differentiation and osteogenic activity of human osteoblasts.

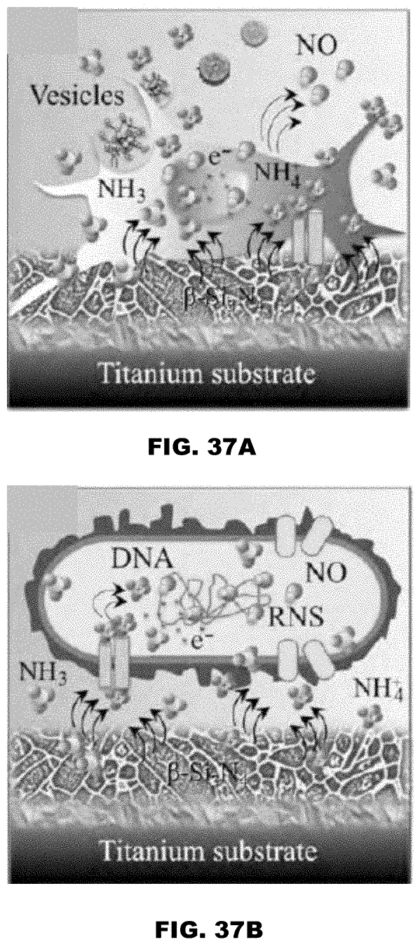

The Si₃N₄ coating showed concurrent osteogenic and antibacterial behaviors like those seen in bulk Si₃N₄ implants. FIGS. 37A and 37B schematically show the chemical interactions of mesenchymal cells and bacteria with Si₃N₄, respectively. The present disclosure shows that the dual behavior of the intrinsic surface chemistry of bulk Si₃N₄ can be replicated on Ti-alloy with a laser-deposited coating.

Comparison with Other Surface Functionalization Technologies

Other coating technologies have targeted antibacterial attributes in arthroplasty implants, and a classification has been proposed, as follows: (i) Passive surface finishing or modification, in which passive coatings (i.e., not releasing bactericidal agents into the surrounding tissues) simply prevent or reduce bacterial adhesion through surface chemistry or structural modifications; (ii) Active surface finishing or modification, in which active coatings feature pharmacologically active pre-incorporated bactericidal agents; and, (iii) Local carriers or coatings (biodegradable or not) that are applied at the time of the surgical procedure around the sample.

The bactericidal effect of Ti-alloy relies on a surface passivation layer, and thus falls into category (i) of “passive” coatings. Ultraviolet light irradiation and partial modification of the crystalline structure (from rutile into anatase phase) of the TiO₂ surface oxide layer can inhibit bacterial adhesion without compromising osteogenesis on titanium alloy implants. Both are relevant to the acetabular shells, since the shells possessed a fraction of anatase phase in their passivation layer, and were UV irradiated for sterilization prior to bacterial testing.

The ability of the Ti-alloy passivation layer to resist bacterial adhesion is limited. It depends on the bacterial species and loads. The long-term effects on the osteogenic behavior of host cells remains a point of concern due to the release of metallic ions. These experiments confirmed the efficacy of the Ti-alloy passivation layer against Gram-positive S. epidermidis but also a lesser effectiveness against Gram-negative E. coli (cf. FIGS. 34A-34J and 36A-36D). In addition, Ti-alloy showed relatively poor in vitro osteoblast activity and quality on flat substrates (cf. FIGS. 33A-33H).

A number of different coatings have been proposed that release pre-incorporated pharmacologically active bactericidal agents, such as metallic ions (e.g., silver, copper, or zinc), non-metallic ions (e.g., iodine or selenium), and organic molecules (e.g., antibiotics, anti-infective peptides, and nitric oxide). These approaches are limited by the cytotoxicity of released ions. Coatings capable of releasing organic molecules such as antibiotics, peptides, and nitric oxide may also be effective against a wide range of pathogens. However, this approach is limited by the duration of efficacy and unpredictable elution kinetics of the pharmacologically active agents. The reliance on released doses of antibiotics also invokes concerns regarding the possible induction of bacterial resistance.

Si₃N₄ coatings on Ti-alloys may confer resistance against adhesion of both Gram-positive and Gram-negative bacteria, while stimulating osteoblasts to deposit high quality bone tissue. Unlike other NO donors, the Si₃N₄ coating released nitrogen under favorable kinetics.

The laser-cladding manufacturing process deposited a dense, tenaciously adherent Si₃N₄ coating (with thickness 10-20 μm) onto porous T-alloy surfaces and commercially available acetabular components. This coating may achieve rapid osseous fixation, while resisting bacteria. Competing technologies have addressed either periprosthetic infections of enhanced osteogenesis, but not both. The dual benefits of a Si₃N₄ coating relate to its RNS surface chemistry, and slow kinetics that favor osseointegration, while inhibiting bacteria.

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A method for coating a metal substrate with a silicon nitride ceramic coating, the method comprising:
providing silicon nitride powder within a laser cladding system having a build platform and a laser beam source;
providing a metal substrate on the build platform, wherein the metal substrate;
providing a constant flow of a gas within the laser cladding system;
spreading a layer of a Si₃N₄ powder over the metal substrate;
pulsing a laser beam from the laser source over the metal substrate and the layer of Si₃N₄ powder to form a silicon nitride coating on the metal substrate; and
repeating the spreading and pulsing steps until the silicon nitride coating has a continuous thickness of 15±5 μm.

2. The method of claim 1, wherein the Si₃N₄ powder comprises a mixture of:
90 wt. % silicon nitride (β-Si₃N₄) which has been previously reacted with 6 wt. % yttrium oxide (Y₂O₃) and 4 wt. % aluminum oxide (Al₂O₃) to form a β-SiYAlON composite structure

3. The method of claim 1, wherein the metal comprises titanium.

4. The method of claim 1, wherein the metal comprises biomedical grade commercially pure titanium.

5. The method of claim 1, wherein the gas comprises nitrogen gas.

6. The method of claim 5, wherein the nitrogen gas limits silicon nitride oxidation and decomposition.

7. The method of claim 1, wherein the laser beam has a wavelength of 1064 nm.

8. The method of claim 1, wherein the laser beam is pulsed with a spot size of 2 mm.

9. The method of claim 1, wherein the laser beam is driven by an applied voltage of 400 V.

10. The method of claim 1, wherein the laser beam is pulsed at a pulse time of 4 ms.

11. The method of claim 1, wherein the silicon nitride coating has greater osteogenic activity than the metal substrate without the silicon nitride coating.

12. The method of claim 1, wherein the silicon nitride coating has greater antibacterial properties than the metal substrate without the silicon nitride coating.
